(19) United States
(12) Patent Application Publication
(10) Pub. No.: US 2021/0115155 A1
(43) Pub. Date: Apr. 22, 2021

(54) METHODS OF TREATING CHRONIC SPONTANEOUS URTICARIA USING LIGELIZUMAB

(71) Applicant: Novartis AG, Basel (CH)

(72) Inventors: Reinhold Martin JANOCHA, Freiburg (DE); Stephan KOEHNE-VOSS, Muttenz (CH); Philip John LOWE, Ipstones (GB); Thomas SEVERIN, Freiburg (DE)

(21) Appl. No.: 17/041,291

(22) PCT Filed: Mar. 25, 2019
(86) PCT No.: PCT/IB2019/052408

§ 371 Date: Sep. 24, 2020

Related U.S. Application Data
(60) Provisional application No. 62/647,928, filed on Mar. 26, 2018.
(60) Provisional application No. 62/811,800, filed on Feb. 28, 2019.

(51) Int. Cl. C07K 16/42; A61P 17/00; A61K 9/00

(52) U.S. Cl. CPC C07K 16/4291; A61K 2039/505; A61K 9/0019; A61P 17/00

(57) ABSTRACT

The present disclosure relates to methods for treating Chronic Spontaneous Urticaria using IgE antagonists, e.g., ligelizumab. Also disclosed herein are IgE antagonists, e.g., IgE antibodies, such as ligelizumab, for treating Chronic Spontaneous Urticaria patients, as well as medicaments, dosing regimens, pharmaceutical formulations, dosage forms, and kits for use in the disclosed uses and methods.

### TECHNICAL FIELD

The present disclosure relates to methods for treating chronic spontaneous urticaria using ligelizumab.

### BACKGROUND OF THE DISCLOSURE

Urticaria is a heterogeneous group of diseases characterized by itchy hives and/or angioedema. Chronic urticaria is defined as urticaria that has been continuously or intermittently present for more than 6 weeks (Maurer M, Magerl M, Metz M, et al (2013) Revisions to the international guidelines on the diagnosis and therapy of chronic urticaria. J Dtsch Dermatol Ges.; Bernstein J A, Lang D M, Khan D A, et al (2014) The diagnosis and management of acute and chronic urticaria: 2014 update. J Allergy Clin Immunol; 133(5):1270-7). Chronic urticaria is then further divided into two subgroups: Chronic Spontaneous Urticaria (CSU) and Inducible Urticaria (IU) the latter including physical urticaria such as heat-, cold-, or pressure-urticaria, and special variants such as cholinergic urticaria. CSU is defined as spontaneous appearance of itchy wheals, angioedema, or both 6 weeks due to known or unknown causes (Zuberbier T, Aberer W, Asero R, et al (2014) The EAACI/GA(2) LEN/EDF/WAO Guideline for the definition, classification, diagnosis, and management of urticaria: the 2013 revision and update. Allergy; 69(7):868-87). A combination of both the CSU and an inducible form of urticaria is possible, such as the frequently observed combination of asymptomatic dermographic urticaria and CSU.

Previously, all chronic urticaria forms without a known trigger were named “chronic idiopathic urticaria” (CIU). Due to medical progress, it is now known that in some of the previously considered “idiopathic” urticaria forms in fact auto-antibodies may be detected. However, the daily fluctuating appearance of the symptoms in this chronic urticaria with auto-antibodies still remains unpredictable and is not induced by a demonstrable trigger, hence, symptoms appear spontaneously. In order to reflect in the terminology correctly that some of the former “idiopathic” forms in fact may have detectable auto-antibodies, this population is now referred to as chronic spontaneous urticaria (CSU) according to the international guideline (Maurer M, Magerl M, Metz M, et al (2013) Revisions to the international guidelines on the diagnosis and therapy of chronic urticaria. J Dtsch Dermatol Ges.; Zuberbier T, Aberer W, Asero R, et al (2014) The EAACI/GA(2) LEN/EDF/WAO Guideline for the definition, classification, diagnosis, and management of urticaria: the 2013 revision and update. Allergy; 69(7):868-87). The use of the expression “chronic idiopathic urticaria” in medical practice is no longer recommended. However, this new naming convention is not implemented in all parts of the world, and in countries such as the United States the patient population with chronic urticaria with a non-specific etiology, or unknown triggers is still referred to as chronic idiopathic urticaria (CIU). Following the International Guideline, the disease entity will be referred to as CSU throughout this document for consistency.

The lifetime prevalence of CSU is approximately 1.8%, and 20% of CSU patients still have the disease after 20 years (Greaves M (2000) Chronic urticaria. J Allergy Clin Immunol; 105(4):664-72; Zuberbier T, Balke M, Worm M, et al (2010) Epidemiology of urticaria: a representative cross-sectional population survey. Clin Exp Dermatol; 35(8):869-73). Affected patients experience frequent pruritic hives with associated erythema and/or episodes of angioedema. Angioedema is reported to be associated with approximately 33-67% of CSU cases (Juhlin L (1981) Recurrent urticaria: clinical investigation of 330 patients. Br J Dermatol; 104(4):369-81; Toubi E, Kessel A, Avshovich N, et al (2004) Clinical and laboratory parameters in predicting chronic urticaria duration: a prospective study of 139 patients. Allergy; 59(8):869-73; Zuberbier T, Balke M, Worm M, et al (2010) Epidemiology of urticaria: a representative cross-sectional population survey. Clin Exp Dermatol; 35(8):869-73; Maurer M, Weller K, Bindslev-Jensen C, et al (2011) Unmet clinical needs in chronic spontaneous urticaria. A GA²LEN task force report. Allergy; 66(3):317-30). The classic skin lesion in urticaria is a wheal and flare with a pale elevated lesion and surrounding erythema, ranging in size from a few millimeters to a few centimeters across, usually occurring in groups and often coalescing to form large confluent lesions. CSU is associated with intense itching and has a major impact on patient well-being and quality of life, suggested to be comparable to that of severe coronary artery disease (Greaves M W (2003) Chronic idiopathic urticaria. Curr Opin Allergy Clin Immunol; 3(5):363-8. Review; Powell R J, Du Toit G L, Siddique N, et al (2007) BSACI guidelines for the management of chronic urticaria and angio-oedema. Clin Exp Allergy; 37(5):631-50). The symptoms of urticaria and urticaria-associated angioedema adversely affect daily activities and sleep (O'Donnell B F, Lawlor F, Simpson J, et al (1997). The impact of chronic urticaria on the quality of life. Br J Dermatol; 136(2):197-201). Therefore, when managing patients with urticaria, patient-related outcomes (e.g., DLQI) are important measures of treatment (Kaplan A, Ledford D, Ashby M, et al (2013) Omalizumab in patients with symptomatic chronic idiopathic/spontaneous urticaria despite standard combination therapy. J Allergy Clin Immunol; 132(1):101-9; Maurer M, Magerl M, Metz M, et al (2013) Revisions to the international guidelines on the diagnosis and therapy of chronic urticaria. J Dtsch Dermatol Ges; Zuberbier T, Aberer W, Asero R, et al (2014) The EAACI/GA(2) LEN/EDF/WAO Guideline for the definition, classification, diagnosis, and management of urticaria: the 2013 revision and update. Allergy; 69(7):868-87).

The pathogenesis of CSU is not fully clear. Up to 50% of CSU cases are associated with histamine-releasing autoantibodies against multiple antigens including the high-affinity IgE receptor (FcεRI) or IgE antibodies; the clinical significance of these autoantibodies is unclear, though there are suggestions that they may be involved in disease pathogenesis (Kaplan A P (2002) Chronic urticaria—new concepts regarding pathogenesis and treatment. Curr Allergy Asthma Rep; 2(4):263-4; Sabroe R A, Greaves M W (2006) Chronic idiopathic urticaria with functional autoantibodies: 12 years on. Br J Dermatol; 154(5):813-9. Review). It has also been suggested that CSU patients' basophils may have distinct alterations in FcεRIα-mediated degranulation, independent of any potential role of autoantibodies (Eckman J A, Hamilton R G, Gober L M, et al (2008) Basophil phenotypes in chronic idiopathic urticaria in relation to disease activity and autoantibodies. J Invest Dermatol; 128(8):1956-63).

Treatment of CSU is a challenge and non-sedating (second generation) H1-antihistamines (H1-AH) are the mainstay of symptomatic therapy of CSU. While H1-AH at approved doses provide relief for some patients, more than 50% of patients do not respond to H1-AH at regular doses. Even when up-dosing to fourfold of the approved dose according to the second step of the treatment algorithm of the current International Guideline (Zuberbier T, Aberer W, Asero R, et al (2014) The EAACI/GA(2) LEN/EDF/WAO Guideline for the definition, classification, diagnosis, and management of urticaria: the 2013 revision and update. Allergy; 69(7):868-87), a substantial part of patients do not experience control of urticaria symptoms (Maurer M, Weller K, Bindslev-Jensen C, et al (2011) Unmet clinical needs in chronic spontaneous urticaria. A GA²LEN task force report. Allergy; 66(3):317-30; Marrouche N, Grattan C (2014) Update and insights into treatment options for chronic spontaneous urticaria. Expert Rev Clin Immunol; 10(3):397-403). For patients without disease control at fourfold doses of H1-AH, the third step of the treatment algorithm of the International Guideline foresees the addition of omalizumab, or ciclosporin A, or montelukast to the H1-AH.

The level of evidence for the efficacy of leukotriene receptor antagonists (LTRA) in urticaria is low but best for montelukast that consequently led to only a weak recommendation from experts for this off-label treatment. Short courses (max. 10 days) of systemic corticosteroids can be added to the 3rd level treatment regimens, if exacerbations demand this. Due to the adverse effects associated with chronic systemic corticosteroid exposure, a longer duration of treatment is not advisable. Other treatment options that were previously used such as intravenous immunoglobulin G, dapsone, hydroxychloroquine, H2-antihistamines (H2-AH), methotrexate, and cyclophosphamide, have an unfavorable benefit risk profile or significant side-effect profile and are no longer recommended for therapy of CSU (Kaplan A P (2002) Chronic urticaria—new concepts regarding pathogenesis and treatment. Curr Allergy Asthma Rep; 2(4):263-4; Powell R J, Du Toit G L, Siddique N, et al (2007) BSACI guidelines for the management of chronic urticaria and angio-oedema. Clin Exp Allergy; 37(5):631-50; Zuberbier T, Aberer W, Asero R, et al (2014) The EAACI/GA(2) LEN/EDF/WAO Guideline for the definition, classification, diagnosis, and management of urticaria: the 2013 revision and update. Allergy; 69(7):868-87).

Omalizumab is an approved therapy for treatment of CSU refractory to standard of care treatment, and exhibits a favorable benefit-risk profile. It is a recombinant humanized IgG₁ monoclonal antibody that binds to IgE-specific epitopes within the C3 (FcεRI binding) region of the IgE molecule and is indicated in many countries for the treatment of poorly controlled moderate or severe asthma and CSU refractory to standard therapy. The completed Phase 2 and Phase 3 studies demonstrate that omalizumab improves the signs and symptoms of urticaria (e.g., itch, hives) in patients with CSU who have failed treatment with H1-AH as well as those who have failed treatment with a combination of H1 and H2-AH and a leukotriene receptor antagonist (Gober M D, Fishelevich R, Zhao Y, et al (2008) Human natural killer T cells infiltrate into the skin at elicitation sites of allergic contact dermatitis. J Invest Dermatol; 128(6):1460-9; Kaplan A P, Joseph K, Maykut R J, et al (2008) Treatment of chronic autoimmune urticaria with omalizumab. J Allergy Clin Immunol; 122(3):569-73; Kaplan A P, Joseph K, Maykut R J, et al (2008) Treatment of chronic autoimmune urticaria with omalizumab. J Allergy Clin Immunol; 122(3):569-73; Kaplan A, Ledford D, Ashby M, et al (2013) Omalizumab in patients with symptomatic chronic idiopathic/spontaneous urticaria despite standard combination therapy. J Allergy Clin Immunol; 132(1):101-9; Maurer M, Magerl M, Metz M, et al (2013) Revisions to the international guidelines on the diagnosis and therapy of chronic urticaria. J Dtsch Dermatol Ges). Published data from the omalizumab Phase 3 study (Q4882g) demonstrated that omalizumab safely improved the clinical manifestations of CSU (e.g., itch, hives), compared to placebo, with monthly doses of 150 mg or 300 mg in a dose-dependent way, but not a 75 mg dose (Kaplan A, Ledford D, Ashby M, et al (2013) Omalizumab in patients with symptomatic chronic idiopathic/spontaneous urticaria despite standard combination therapy. J Allergy Clin Immunol; 132(1):101-9; Maurer M, Magerl M, Metz M, et al (2013) Revisions to the international guidelines on the diagnosis and therapy of chronic urticaria. J Dtsch Dermatol Ges.). The primary endpoint, the mean (±SD) change from baseline in the weekly itch-severity score at Week 12, was −5.1±5.6 in the placebo group, −5.9±6.5 in the 75 mg group (P=0.46), −8.1±6.4 in the 150 mg group (P=0.001), and −9.8±6.0 in the 300 mg group (P<0.001) and all pre-specified secondary end points (e.g., change from baseline in UAS7, weekly score for hive numbers, proportion of patients with UAS7≤6) were dose-responsive. The exact mechanism for how omalizumab may work for CSU patients is unknown.

QGE031 (ligelizumab) is a humanized monoclonal antibody with higher affinity binding to human immunoglobulin E (IgE) than omalizumab. Upon binding, QGE031 is able to block the interaction of IgE with both the high and low affinity IgE receptors (FcεRI and FcεRII). QGE031 is unable to mediate IgE receptor cross-linking and consequently histamine release (i.e. non-anaphylactogenic). When patients receive QGE031, circulating IgE is rapidly bound by the anti-IgE antibody and becomes inaccessible to IgE receptors on mast cells and basophils. IgE is necessary for the enhanced expression of the FcεRI seen in atopic patients, and a decrease in FcεRI expression on circulating basophils accompanies QGE031 treatment. Other potentially beneficial effects from anti-IgE therapy include decreased IgE production, reduced B cell numbers and reduced cytokine production by T cells.

QGE031 demonstrated dose- and time-dependent suppression of free IgE, basophil FcεRI, basophil surface IgE, and skin prick test responses to allergen, superior in extent and duration to those observed with omalizumab. Superior affinity and pharmacodynamic (PD) outcomes of QGE031 compared to omalizumab may translate into superior posology and superior clinical efficacy in patients with CSU (see FIG. 2).

### SUMMARY OF THE DISCLOSURE

We have now devised regimens for treating CSU patients with ligelizumab or antigen-binding fragments thereof that are remarkably effective and provide sustained responses for CSU patients. Accordingly, disclosed herein are methods of treating chronic spontaneous urticaria (CSU), comprising subcutaneously (SC) administering to a patient in need thereof a dose of about 24 mg-about 240 mg of ligelizumab antibody, or an antigen binding fragment thereof, during week 0, and thereafter SC at a dose of about 24 mg-about 240 mg monthly (every 4 weeks), beginning during week 4;

wherein the ligelizumab antibody or antigen binding fragment thereof comprises:

an immunoglobulin V_{H} domain comprising the amino acid sequence set forth as SEQ ID NO: 62 and an immunoglobulin V_{L} domain comprising the amino acid sequence set forth as SEQ ID NO:1 SEQ ID NO:1.

### BRIEF DESCRIPTION OF THE FIGURES

FIG. 1 shows the ligelizumab CSU clinical trial design.

FIG. 2 Panels show individual patient data of B2203 for different treatment arms (Placebo, 24, 72, 240 mg QGE031, 300 mg Xolair/omalizumab) and for different biomarkers and skin prick test results. Patients with IgE level >250 IU/ml are shown with dashed lines connecting triangle symbols. The labels ‘FceR1’ and ‘sIgE’ are basophil Fc epsilon receptor type 1 and surface bound IgE; the units are molecules of equivalent soluble fluorophore (MESF). ‘Wheal’ refers to the size of the allergen induced wheals in skin prick testing; this is the sum of the data from all dilutions, plotted on a square root scale to correctly represent the statistical distribution.

FIG. 3 shows the predicted dose response curves for QGE031 for the wheal component of the skin prick test. A 24 mg dose was predicted to achieve 50-70% of the maximal possible response. The range of responses for the middle 50% of the patient population spans from a very small to a high but not maximal response. A dose of 72 mg presumably was close to the transition between the linear and the saturated region of the dose response curve while 240 mg was anticipated to achieve maximal efficacy. Therefore, 24 mg represents a ‘sub optimal dose’ that was anticipated to be in the same range as omalizumab, rather than a minimally effective dose. Omalizumab 300 mg q4w is predicted to give a response slightly below that for QGE031 72 mg q4w.

FIG. 4 shows the effect of 24, 72 and 240 mg multiple administration and a single 120 mg dose of QGE031 on UAS7 change from baseline. The x-axis is time in weeks, the y-axis is UAS7 change from baseline and the shaded bands are 80% confidence intervals. It can be seen that for the first 4-6 weeks there is no significant difference between the patients' response to 72, 120 or 240 mg QGE031. Only the 24 mg dose shows lesser efficacy, but even this dose of QGE031 is better than the control placebo treatment.

### DETAILED DESCRIPTION OF THE DISCLOSURE

Ligelizumab, also known as QGE031, is a generally Y-shaped tetrameric molecule having an antigen binding site at the end of each upper arm. The antigen binding site is formed by three complementarity determining regions (CDRs) of the variable region of a heavy chain (VH) and three CDRs of the variable region of a light chain (VL). In both VH and VL the CDRs alternate with four framework regions (FRs) forming a polypeptide chain of general formula FR-CDR1-FR2-a CDR2-FR3-CDR3-FR4. Ligelizumab is disclosed in U.S. Pat. No. 7,531,169 as Mab 2 (CL-2C) and defined by SEQ ID NOs: 61 and 62, which is incorporated by reference herein in its entirety.

The term “comprising” encompasses “including” as well as “consisting,” e.g., a composition “comprising” X may consist exclusively of X or may include something additional, e.g., X+Y.

The term “about” in relation to a numerical value x means, for example, +/−10%. When used in front of a numerical range or list of numbers, the term “about” applies to each number in the series, e.g., the phrase “about 1-5” should be interpreted as “about 1-about 5”, or, e.g., the phrase “about 1, 2, 3, 4” should be interpreted as “about 1, about 2, about 3, about 4, etc.”

The word “substantially” does not exclude “completely,” e.g., a composition which is “substantially free” from Y may be completely free from Y. Where necessary, the word “substantially” may be omitted from the definition of the disclosure.

The term “antibody” as referred to herein includes naturally-occurring and whole antibodies. A naturally-occurring “antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed hypervariable regions or complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. Exemplary antibodies include ligelizumab antibody (U.S. Pat. No. 7,531,169), the disclosure of which is incorporated by reference herein in their entirety.

The term “antigen-binding fragment” of an antibody, as used herein, refers to fragments of an antibody that retain the ability to specifically bind to an antigen (e.g., IgE). It has been shown that the antigen-binding function of an antibody can be performed by fragments of a full-length antibody. Examples of binding fragments encompassed within the term “antigen-binding portion” of an antibody include a Fab fragment, a monovalent fragment consisting of the V_{L}, V_{H}, CL and CH1 domains; a F(ab)2 fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., 1989 Nature 341:544-546), which consists of a V_{H} domain; and an isolated CDR. Exemplary antigen-binding fragments include the CDRs of ligelizumab having a variable light chain region comprising CDRL1, CDRL2, and CDRL3 and a variable heavy chain region comprising CDRH1, CDRH2, and CDRH3, wherein CDRL1 consists of SEQ ID NO:3, CDRL2 consists of SEQ ID NO:4, CDRL3 consists of SEQ ID NO:5, CDRH1 consists of SEQ ID NO:6, CDRH2 consists of SEQ ID NO:7, and CDRH3 consists of SEQ ID NO:8, wherein the antibody binds specifically to IgE.

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al., 1988 Science 242:423-426; and Huston et al., 1988 Proc. Natl. Acad. Sci. 85:5879-5883). Such single chain antibodies are also intended to be encompassed within the term “antibody”. Single chain antibodies and antigen-binding portions are obtained using conventional techniques known to those of skill in the art.

An “isolated antibody”, as used herein, refers to an antibody that is substantially free of other antibodies having different antigenic specificities (e.g., an isolated antibody that specifically binds IgE is substantially free of antibodies that specifically bind antigens other than IgE). The term “monoclonal antibody” or “monoclonal antibody composition” as used herein refer to a preparation of antibody molecules of single molecular composition. The term “human antibody”, as used herein, is intended to include antibodies having variable regions in which both the framework and CDR regions are derived from sequences of human origin. A “human antibody” need not be produced by a human, human tissue or human cell. The human antibodies of the disclosure may include amino acid residues not encoded by human sequences (e.g., mutations introduced by random or site-specific mutagenesis in vitro, by N-nucleotide addition at junctions in vivo during recombination of antibody genes, or by somatic mutation in vivo). In some embodiments of the disclosed processes and compositions, the IgE antibody is a human antibody, an isolated antibody, and/or a monoclonal antibody.

As used herein, “anti-human IgE antibody” means an antibody that binds to human IgE in such a manner so as to inhibit or substantially reduce the binding of such IgE to the high affinity receptor, FcεRI.

The term “affinity” refers to the strength of interaction between antibody and antigen at single antigenic sites. Within each antigenic site, the variable region of the antibody “arm” interacts through weak non-covalent forces with antigen at numerous sites; the more interactions, the stronger the affinity. Standard assays to evaluate the binding affinity of the antibodies toward IgE of various species are known in the art, including for example, ELISAs, western blots and RIAs. The binding kinetics (e.g., binding affinity) of the antibodies also can be assessed by assays known in the art, e.g., K_{D} can be determined using a Biacore® analysis.

An antibody that “inhibits” one or more IgE functional properties (e.g., biochemical, immunochemical, cellular, physiological or other biological activities, or the like) as determined according to methodologies known to the art and described herein, will be understood to relate to a statistically significant decrease in the particular activity relative to that seen in the absence of the antibody (or when a control antibody of irrelevant specificity is present). An antibody that inhibits IgE activity affects a statistically significant decrease, e.g., by at least about 10% of the measured parameter, by at least 50%, 80% or 90%, and in certain embodiments of the disclosed methods and compositions, the IgE antibody used may inhibit greater than 95%, 98% or 99% of IgE functional activity.

The term “derivative”, unless otherwise indicated, is used to define amino acid sequence variants, and covalent modifications (e.g., pegylation, deamidation, hydroxylation, phosphorylation, methylation, etc.) of an IgE antibody or antigen-binding fragment thereof, e.g., ligelizumab, according to the present disclosure, e.g., of a specified sequence (e.g., a variable domain). A “functional derivative” includes a molecule having a qualitative biological activity in common with the disclosed IgE antibodies. A functional derivative includes fragments and peptide analogs of an IgE antibody as disclosed herein. Fragments comprise regions within the sequence of a polypeptide according to the present disclosure, e.g., of a specified sequence. Functional derivatives of the IgE antibodies disclosed herein (e.g., functional derivatives of ligelizumab) preferably comprise V_{H} and/or V_{L} domains that have at least about 65%, 75%, 85%, 95%, 96%, 97%, 98%, or even 99% overall sequence identity with the V_{H} and/or V_{L} sequences of the IGE antibodies and antigen-binding fragments thereof disclosed herein and substantially retain the ability to bind human IgE.

The phrase “substantially identical” means that the relevant amino acid or nucleotide sequence (e.g., V_{H} or V_{L} domain) will be identical to or have insubstantial differences (e.g., through conserved amino acid substitutions) in comparison to a particular reference sequence. Insubstantial differences include minor amino acid changes, such as 1 or 2 substitutions in a 5 amino acid sequence of a specified region (e.g., V_{H} or V_{L} domain). In the case of antibodies, the second antibody has the same specificity and has at least 50% of the affinity of the same. Sequences substantially identical (e.g., at least about 85% sequence identity) to the sequences disclosed herein are also part of this application. In some embodiments, the sequence identity of a derivative IgE antibody (e.g., a derivative of ligelizumab, e.g., a ligelizumab biosimilar antibody) can be about 90% or greater, e.g., 90%, 91%, 92%, 93%, 94%, 95%, 96%, 97%, 98%, 99% or higher relative to the disclosed sequences.

“Identity” with respect to a native polypeptide and its functional derivative is defined herein as the percentage of amino acid residues in the candidate sequence that are identical with the residues of a corresponding native polypeptide, after aligning the sequences and introducing gaps, if necessary, to achieve the maximum percent identity, and not considering any conservative substitutions as part of the sequence identity. Neither N- or C-terminal extensions nor insertions shall be construed as reducing identity. Methods and computer programs for the alignment are known. The percent identity can be determined by standard alignment algorithms, for example, the Basic Local Alignment Search Tool (BLAST) described by Altshul et al. ((1990) J. Mol. Biol., 215: 403 410); the algorithm of Needleman et al. ((1970) J. Mol. Biol., 48: 444 453); or the algorithm of Meyers et al. ((1988) Comput. Appl. Biosci., 4: 11 17). A set of parameters may be the Blosum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

“Amino acid(s)” refer to all naturally occurring L-α-amino acids, e.g., and include D-amino acids. The phrase “amino acid sequence variant” refers to molecules with some differences in their amino acid sequences as compared to the sequences according to the present disclosure.

Amino acid sequence variants of an antibody according to the present disclosure, e.g., of a specified sequence, still have the ability to bind the human IgE. Amino acid sequence variants include substitutional variants (those that have at least one amino acid residue removed and a different amino acid inserted in its place at the same position in a polypeptide according to the present disclosure), insertional variants (those with one or more amino acids inserted immediately adjacent to an amino acid at a particular position in a polypeptide according to the present disclosure) and deletional variants (those with one or more amino acids removed in a polypeptide according to the present disclosure).

The term “pharmaceutically acceptable” means a nontoxic material that does not interfere with the effectiveness of the biological activity of the active ingredient(s).

The term “administering” in relation to a compound, e.g., an IgE binding molecule or another agent, is used to refer to delivery of that compound to a patient by any route.

As used herein, a “therapeutically effective amount” refers to an amount of an IgE antagonist, e.g., IgE binding molecule (e.g., IgE antibody or antigen-binding fragment thereof, e.g., ligelizumab) or IgE receptor binding molecule (e.g., IgE antibody or antigen-binding fragment thereof) that is effective, upon single or multiple dose administration to a patient (such as a human) for treating, preventing (if applicable), preventing the onset of (if applicable), curing, delaying, reducing the severity of, ameliorating at least one symptom of a disorder or recurring disorder, or prolonging the survival of the patient beyond that expected in the absence of such treatment. When applied to an individual active ingredient (e.g., an IgE antagonist, e.g., ligelizumab) administered alone, the term refers to that ingredient alone. When applied to a combination, the term refers to combined amounts of the active ingredients that result in the therapeutic effect, whether administered in combination, serially or simultaneously.

The term “treatment” or “treat” is herein defined as the application or administration of an IgE antibody according to the disclosure, for example ligelizumab or a pharmaceutical composition comprising said anti-IgE antibody, to a subject or to an isolated tissue or cell line from a subject, where the subject has a particular disease (e.g., CSU), a symptom associated with the disease (e.g., CSU), or a predisposition towards development of the disease (e.g., CSU) (if applicable), where the purpose is to cure (if applicable), delay the onset of, reduce the severity of, alleviate, ameliorate one or more symptoms of the disease, improve the disease, reduce or improve any associated symptoms of the disease or the predisposition toward the development of the disease. The term “treatment” or “treat” includes treating a patient suspected to have the disease as well as patients who are ill or who have been diagnosed as suffering from the disease or medical condition, and includes suppression of clinical relapse.

As used herein, the phrase “population of patients” is used to mean a group of patients. In some embodiments of the disclosed methods, the IgE antagonist (e.g., IgE antibody, such as ligelizumab) is used to treat a population of CSU patients.

As used herein, the phrases “has not been previously treated with a systemic treatment for CSU” and “naïve” refer to a CSU patient who has not been previously treated with a systemic agent, e.g., ciclosporin A, montelukast, H1-antihistamines (H1-AH), H2-AH, and a leukotriene receptor antagonist (LTRA), a biological (e.g., omalizumab.), etc., for CSU. Systemic agents (i.e., agents given orally, by injection, etc.) differ from local agents (e.g., topicals and phototherapy) in that systemic agents have a systemic (whole body) effect when delivered to a patient. In some embodiments of the disclosed methods, regimens, uses, kits, and pharmaceutical compositions, the patient has not been previously administered a systemic treatment for CSU.

As used herein, the phrase “has been previously treated with a systemic agent for CSU” is used to mean a patient that has previously undergone CSU treatment using a systemic agent. Such patients include those previously treated with biologics, such as omalizumab, and those previously treated with non-biologics, such as cyclosporine. In some embodiments of the disclosure, the patient has been previously administered a systemic agent for CSU. In some embodiments, the patient has been previously administered a systemic agent for CSU (e.g., cyclosporine), but the patient has not been previously administered a systemic biological drug (i.e., a drug produced by a living organism, e.g., antibodies, receptor decoys, etc.) for CSU (e.g., omalizumab). In this case, the patient is referred to as “biological-naïve.” In some embodiments, the patient is biological-naïve.

As used herein, “selecting” and “selected” in reference to a patient is used to mean that a particular patient is specifically chosen from a larger group of patients on the basis of (due to) the particular patient having a predetermined criteria. Similarly, “selectively treating” refers to providing treatment to a patient having a particular disease, where that patient is specifically chosen from a larger group of patients on the basis of the particular patient having a predetermined criterion. Similarly, “selectively administering” refers to administering a drug to a patient that is specifically chosen from a larger group of patients on the basis of (due to) the particular patient having a predetermined criterion. By selecting, selectively treating and selectively administering, it is meant that a patient is delivered a personalized therapy based on the patient's personal history (e.g., prior therapeutic interventions, e.g., prior treatment with biologics), biology (e.g., particular genetic markers), and/or manifestation (e.g., not fulfilling particular diagnostic criteria), rather than being delivered a standard treatment regimen based solely on the patient's membership in a larger group. Selecting, in reference to a method of treatment as used herein, does not refer to fortuitous treatment of a patient having a particular criterion, but rather refers to the deliberate choice to administer treatment to a patient based on the patient having a particular criterion. Thus, selective treatment/administration differs from standard treatment/administration, which delivers a particular drug to all patients having a particular disease, regardless of their personal history, manifestations of disease, and/or biology. In some embodiments, the patient was selected for treatment based on having CSU.

### IgE Antagonists

The various disclosed processes, kits, uses and methods utilize an IgE antagonist, e.g., IgE binding molecule (e.g., soluble IgE receptor, IgE antibody or antigen-binding fragment thereof, e.g., ligelizumab) or IgE receptor binding molecule (e.g., IgE receptor antibody or antigen-binding fragment thereof). In some embodiments, the IgE antagonist is an IgE binding molecule, preferably an IgE antibody or antigen-binding fragment thereof.

In one embodiment, the IgE antibody or antigen-binding fragment thereof comprises an immunoglobulin VH domain comprising the amino acid sequence set forth as SEQ ID NO:2 and an immunoglobulin VL domain comprising the amino acid sequence set forth as SEQ ID NO:1 SEQ ID NO:1.

In one embodiment, the IgE antibody or antigen-binding fragment thereof comprises a variable light chain region comprising CDRL1, CDRL2, and CDRL3 and a variable heavy chain region comprising CDRH1, CDRH2, and CDRH3, wherein CDRL1 consists of SEQ ID NO:3, CDRL2 consists of SEQ ID NO:4, CDRL3 consists of SEQ ID NO:5, CDRH1 consists of SEQ ID NO:6, CDRH2 consists of SEQ ID NO:7, and CDRH3 consists of SEQ ID NO:8, wherein the antibody binds specifically to IgE.

Alternatively, an IgE antibody or antigen-binding fragment thereof as used in the disclosed methods may comprise a derivative of the IgE antibodies set forth herein by sequence (e.g., pegylated variants, glycosylation variants, affinity-maturation variants, etc.). Alternatively, the V_{H} or V_{L} domain of an IgE antibody or antigen-binding fragment thereof used in the disclosed methods may have V_{H} or V_{L} domains that are substantially identical to the V_{H} or V_{L} domains set forth herein (e.g., those set forth in SEQ ID NO:2 and 61). A human IgE antibody disclosed herein may comprise a heavy chain that is substantially identical to that set forth as SEQ ID NO:2 and/or a light chain that is substantially identical to that set forth as SEQ ID NO:1. A human IgE antibody disclosed herein may comprise a heavy chain that comprises SEQ ID NO:2 and a light chain that comprises SEQ ID NO:1.

The preferred IgE antibodies or antigen-binding fragments thereof used in the disclosed methods are human antibodies, especially ligelizumab as described in Table 2 of Examples 10 of U.S. Pat. No. 7,531,169, which is incorporated by reference herein in its entirety.

### Methods of Treatment and Uses of IgE Antagonists for CSU

The disclosed IgE antagonists, e.g., IgE binding molecules (e.g., IgE antibody or antigen-binding fragment thereof, e.g., ligelizumab) or IgE receptor binding molecules (e.g., IgE receptor antibody or antigen-binding fragment thereof), may be used in vitro, ex vivo, or incorporated into pharmaceutical compositions and administered in vivo to treat CSU patients (e.g., human patients).

Urticaria is a heterogeneous group of diseases characterized by itchy hives and/or angioedema.

Chronic urticaria is defined as urticaria that has been continuously or intermittently present for more than 6 weeks (Maurer, et al 2013, Bernstein, et al 2014). Chronic urticaria is then further divided into two subgroups: Chronic Spontaneous Urticaria (CSU) and Inducible Urticaria (IU) the latter including physical urticaria such as heat-, cold-, or pressure-urticaria, and special variants such as cholinergic urticaria. CSU is defined as spontaneous appearance of itchy wheals, angioedema, or both ≥6 weeks due to known or unknown causes (Zuberbier, et al 2014). A combination of both the CSU and an inducible form of urticaria is possible, such as the frequently observed combination of asymptomatic dermographic urticaria and CSU.

The effectiveness of a CSU treatment may be assessed using various known methods and tools that measure CSU disease state and/or CSU clinical response. Some examples include, e.g., Weekly Hives Severity Score (HSS7), Weekly Itch Severity Score (ISS7), and Weekly Urticaria Activity Score (UAS7).

In some embodiments, the patient is treated for HS according to the claimed methods for at least 20 weeks, at least 48 weeks, at least 52 weeks, or at least 2 years.

In some embodiments, the patient previously had an inadequate response to conventional systemic CSU therapy.

In some embodiments, the patient is an adolescent patient (≥12 years of age) having moderate to severe CSU. In some embodiments, the patient is an adult patient having moderate to severe CSU.

In some embodiments, in response to treatment according to the claimed methods, the patient experiences rapid reduction hives, as measured by HSS7 scoring, as early as 1 week after initial dosing.

The IgE antagonists, e.g., IgE binding molecules (e.g., IgE antibody or antigen-binding fragment thereof, e.g., ligelizumab) or IgE receptor binding molecules (e.g. IgE antibody or antigen-binding fragment thereof), may be used as a pharmaceutical composition when combined with a pharmaceutically acceptable carrier. Such a composition may contain, in addition to an IgE antagonist, carriers, various diluents, fillers, salts, buffers, stabilizers, solubilizers, and other materials known in the art. The characteristics of the carrier will depend on the route of administration. The pharmaceutical compositions for use in the disclosed methods may also contain additional therapeutic agents for treatment of the particular targeted disorder. For example, a pharmaceutical composition may also include anti-inflammatory or anti-itch agents. Such additional factors and/or agents may be included in the pharmaceutical composition to produce a synergistic effect with the IgE binding molecules, or to minimize side effects caused by the IgE antagonists, e.g., IgE binding molecules (e.g., IgE antibody or antigen-binding fragment thereof, e.g., ligelizumab) or IgE receptor binding molecules (e.g., IgE antibody or antigen-binding fragment thereof). In preferred embodiments, the pharmaceutical compositions for use in the disclosed methods comprise ligelizumab at 120 mg/ml.

Pharmaceutical compositions for use in the disclosed methods may be manufactured in conventional manner. In one embodiment, the pharmaceutical composition is provided in lyophilized form. For immediate administration, it is dissolved in a suitable aqueous carrier, for example sterile water for injection or sterile buffered physiological saline. If it is considered desirable to make up a solution of larger volume for administration by infusion rather than a bolus injection, may be advantageous to incorporate human serum albumin or the patient's own heparinized blood into the saline at the time of formulation. The presence of an excess of such physiologically inert protein prevents loss of antibody by adsorption onto the walls of the container and tubing used with the infusion solution. If albumin is used, a suitable concentration is from 0.5 to 4.5% by weight of the saline solution. Other formulations comprise ready-to-use liquid formulations, which may be disposed in, e.g., a vial, syringe, auto-injector, etc.

Antibodies, e.g., antibodies to IgE, are typically formulated either in aqueous form ready for parenteral administration or as lyophilisates for reconstitution with a suitable diluent prior to administration. In preferred embodiments of the disclosed methods and uses, the IgE antagonist, e.g., IgE antibody, e.g., ligelizumab, is formulated as ready to use liquid pharmaceutical formulation. In some embodiments of the disclosed methods and uses, the IgE antagonist, e.g., IgE antibody, e.g., ligelizumab, is formulated as a lyophilisate. Suitable lyophilisate formulations can be reconstituted in a small liquid volume (e.g., 2 mL or less, e.g., 2 mL, 1 mL, etc.) to allow subcutaneous administration and can provide solutions with low levels of antibody aggregation. The use of antibodies as the active ingredient of pharmaceuticals is now widespread, including the products HERCEPTIN™ (trastuzumab), RITUXAN™ (rituximab), SYNAGIS™ (palivizumab), etc. Techniques for purification of antibodies to a pharmaceutical grade are known in the art. When a therapeutically effective amount of an IgE antagonist, e.g., IgE binding molecules (e.g., IgE antibody or antigen-binding fragment thereof, e.g., ligelizumab) or IgE receptor binding molecules (e.g., IgE antibody or antigen-binding fragment thereof) is administered by intravenous, cutaneous or subcutaneous injection, the IgE antagonist will be in the form of a pyrogen-free, parenterally acceptable solution. A pharmaceutical composition for intravenous, cutaneous, or subcutaneous injection may contain, in addition to the IgE antagonist, an isotonic vehicle such as sodium chloride, Ringer's solution, dextrose, dextrose and sodium chloride, lactated Ringer's solution, or other vehicle as known in the art.

In practicing some of the methods of treatment or uses of the present disclosure, a therapeutically effective amount of an IgE antagonist, e.g., IgE binding molecule (e.g., IgE antibody or antigen-binding fragment thereof, e.g., ligelizumab) or IgE receptor binding molecule (e.g., IgE antibody or antigen-binding fragment thereof) is administered to a patient, e.g., a mammal (e.g., a human). While it is understood that the disclosed methods provide for treatment of CSU patients using an IgE antagonist (e.g., ligelizumab), this does not preclude that, if the patient is to be ultimately treated with an IgE antagonist, such IgE antagonist therapy is necessarily a monotherapy. Indeed, if a patient is selected for treatment with an IgE antagonist, then the IgE antagonist (e.g., ligelizumab) may be administered in accordance with the methods of the disclosure either alone or in combination with other agents and therapies for treating CSU patients, e.g., in combination with at least one additional CSU agent. When co-administered with one or more additional CSU agent(s), an IgE antagonist may be administered either simultaneously with the other agent, or sequentially. If administered sequentially, the attending physician will decide on the appropriate sequence of administering the IgE antagonist in combination with other agents and the appropriate dosages for co-delivery.

Various therapies may be beneficially combined with the disclosed IgE antibodies, such as ligelizumab, during treatment of CSU. Such therapies include topical treatments (creams [non-steroidal or steroidal], washes, antiseptics,), systemic treatments (e.g., with biologicals, antibiotics, or chemical entities), and antiseptics, photodynamic therapy, and surgical intervention (laser, draining or incision, excision).

Non-limiting examples of topical CSU agents for use with the disclosed IgE antibodies, such as ligelizumab, include benzoyl peroxide, topical steroid creams, topical antibiotics in the aminoglycoside group, such as clindamycin, gentamicin, and erythromycin, resorcinol cream, iodine scrubs, and chlorhexidine.

Non-limiting examples of CSU agents used in systemic treatment for use with the disclosed IgE antibodies, such as ligelizumab, include further IgE antagonists (omalizumab).

Additional CSU agents for use in combination with the disclosed IgE antibodies, such as ligelizumab, during treatment of CSU include cyclosporine and corticosteroids (injectable or oral).

A skilled artisan will be able to discern the appropriate dosages of the above CSU agents for co-delivery with the disclosed IgE antibodies, such as ligelizumab.

An IgE antagonist, e.g., IgE binding molecule (e.g., IgE antibody or antigen-binding fragment thereof, e.g., ligelizumab) or IgE receptor binding molecule (e.g., IgE receptor antibody or antigen-binding fragment thereof) is conveniently administered parenterally, e.g., intravenously (e.g., into the antecubital or other peripheral vein), intramuscularly, or subcutaneously. The duration of intravenous (IV) therapy using a pharmaceutical composition of the present disclosure will vary, depending on the severity of the disease being treated and the condition and personal response of each individual patient. Also contemplated is subcutaneous (SC) therapy using a pharmaceutical composition of the present disclosure. The health care provider will decide on the appropriate duration of IV or SC therapy and the timing of administration of the therapy, using the pharmaceutical composition of the present disclosure. In preferred embodiments, the IgE antagonist (e.g., ligelizumab) is administered via the subcutaneous (SC) route.

The IgE antagonist, e.g., IgE binding molecule (e.g., IgE antibody or antigen-binding fragment thereof, e.g., ligelizumab) or IgE receptor binding molecule (e.g., IgE receptor antibody or antigen-binding fragment thereof) may be administered to the patient intravenously (SC) every four weeks starting at week 0 and 4 and thereafter administered to the patient SC, e.g., at about 24 mg-about 240 mg (e.g., about 24 mg, about 240 mg) every four weeks, beginning during week 4. In this manner, the patient receives a SC dose during week 0, 4, 8, 12, 16, etc.

Alternatively the IgE antagonist, e.g., IgE binding molecule (e.g., IgE antibody or antigen-binding fragment thereof, e.g., ligelizumab) or IgE receptor binding molecule (e.g., IgE receptor antibody or antigen-binding fragment thereof) may be administered to the patient subcutaneously (SC) every four weeks starting at week 0 and thereafter administered to the patient SC, e.g., at about 24 mg, about 72 mg, about 120 mg to about 240 mg (e.g., about 24 mg, about 240 mg) every four weeks, beginning during week 4. In this manner, the patient is dosed SC with about 24 mg, about 72 mg, about 120 mg to about 240 mg (e.g., about 24 mg, about 72 mg, about 120 mg to about 240 mg) of the IgE antagonist (e.g., ligelizumab) during weeks 0, 4, 8, 12, etc.

Preferably, the IgE antagonist, e.g., IgE binding molecule (e.g., IgE antibody or antigen-binding fragment thereof, e.g., ligelizumab) or IgE receptor binding molecule (e.g., IgE receptor antibody or antigen-binding fragment thereof) may be administered to the patient subcutaneously (SC) every four weeks starting at week 0 and thereafter administered to the patient SC, e.g., at about 24 mg, about 72 mg, about 120 mg to about 240 mg (e.g., about 24 mg, about 72 mg, about 120 mg to about 240 mg) every four weeks, beginning during week 4. In this manner, the patient is dosed SC with about 24 mg, about 72 mg, about 120 mg to about 240 mg (e.g., about 24 mg, about 72 mg, about 120 mg to about 240 mg) of the IgE antagonist (e.g., ligelizumab) during weeks 0, 4, 8, 12, 16, 20, etc.

More preferably, the IgE antagonist, e.g., IgE binding molecule (e.g., IgE antibody or antigen-binding fragment thereof, e.g., ligelizumab) or IgE receptor binding molecule (e.g., IgE receptor antibody or antigen-binding fragment thereof) may be administered to the patient without a loading regimen, e.g., the antagonist may be administered to the patient SC at about 24 mg, about 72 mg, about 120 mg to about 240 mg (e.g., about 24 mg, about 72 mg, about 120 mg to about 240 mg) every four weeks. In this manner, the patient is dosed SC with about 24 mg, about 72 mg, about 120 mg to about 240 mg (e.g., about 24 mg, about 72 mg, about 120 mg to about 240 mg) of the IgE antagonist (e.g., ligelizumab) during weeks 0, 4, 8, 12, etc.

It will be understood that dose escalation may be required for certain patients, e.g., CSU patients that display inadequate response (e.g., as measured by any of the CSU scoring systems disclosed herein, e.g., The method according to any of the above claims, wherein said patient achieves a sustained response as measured by complete hives response (Hives Severity Score [HSS7]), UAS7 and Dermatology Life Quality Index (DLQI), etc.) to treatment with the IgE antagonists, e.g., IgE binding molecules (e.g., IgE antibody or antigen-binding fragment thereof, e.g., ligelizumab) or IgE receptor binding molecules (e.g., IgE receptor antibody or antigen-binding fragment thereof) by week 12, week 16, week 20, week 24, week 48 or week 52 of treatment. It will also be understood that dose reduction may also be required for certain patients, e.g., CSU patients that display adverse events or an adverse response to treatment with the IgE antagonist (e.g., IgE antibody or antigen-binding fragment thereof, e.g., ligelizumab). Thus, dosages of the IgE antagonist (e.g., IgE antibody or antigen-binding fragment thereof, e.g., ligelizumab), may be less than about 24 mg, about 72 mg, about 120 mg to about 240 mg SC. In some embodiments, the IgE antagonist, e.g., IgE binding molecule (e.g., IgE antibody or antigen-binding fragment thereof, e.g., ligelizumab) or IgE receptor binding molecule (e.g., IgE receptor antibody or antigen-binding fragment thereof) may be administered to the patient at an initial dose of about 24 mg, about 72 mg, about 120 mg to about 240 mg delivered SC, and the dose is then escalated to about 72 mg (in the case of an original 24 mg dose) or about 240 mg (in the case of an original 120 mg dose) if needed, as determined by a physician.

The timing of dosing is generally measured from the day of the first dose of ligelizumab (which is also known as “baseline”). However, health care providers often use different naming conventions to identify dosing schedules, as shown in Table 1.

TABLE 1
Common naming conventions for dosing regimens.

| Week | 0/1 | 4/5 | 8/9 | 12/11 | 16/15 | 20/19 | etc. |
|---|---|---|---|---|---|---|---|
| 1^{st} day | 0/1 | 28/29 | 56/57 | 84/85 | 112/113 | 140/141 | etc. |
| of week | | | | | | | |

Bolded items refer to the naming convention used herein.

Notably, week zero may be referred to as week one by some health care providers, while day zero may be referred to as day one by some health care providers. Thus, it is possible that different physicians will designate, e.g., a dose as being given during week 4/on day 28, during week 4/on day 29, during week 4/on day 28, during week 4/on day 29, while referring to the same dosing schedule. For consistency, the first week of dosing will be referred to herein as week 0, while the first day of dosing will be referred to as day 1. However, it will be understood by a skilled artisan that this naming convention is simply used for consistency and should not be construed as limiting, i.e., weekly dosing is the provision of a weekly dose of the IgE antibody regardless of whether the physician refers to a particular week as “week 0” or “week 1”.

In a one dosing regimen, the antibody is administered during week 0, 4, 8, 12, 16, 20, etc. Some providers may refer to this regimen as monthly dosing (or dosing every 4 weeks). It will be appreciated by a skilled artisan that administering a patient an injection at weeks 0 followed by once monthly dosing starting at week 4 is the same as: 1) administering the patient an injection at weeks 0 and 4, followed by once monthly dosing starting at week 8; 2) administering the patient an injection at weeks 0 and 4 followed by dosing every 4 weeks; and 3) administering the patient an injection at weeks 0 and 4 followed by monthly administration.

As used herein, the phrase “formulated at a dosage to allow [route of administration] delivery of [a designated dose]” is used to mean that a given pharmaceutical composition can be used to provide a desired dose of an IgE antagonist, e.g., an IgE antibody, e.g., ligelizumab, via a designated route of administration (e.g., SC or IV). As an example, if a desired SC dose is 240 mg, then a clinician may use 2 ml of an IgE antibody formulation having a concentration of 120 mg/ml, 1 ml of an IgE antibody formulation having a concentration of 240 mg/ml, 0.5 ml of an IgE antibody formulation having a concentration of 480 mg/ml, etc. In each such case, these IgE antibody formulations are at a concentration high enough to allow subcutaneous delivery of the IgE antibody. Subcutaneous delivery typically requires delivery of volumes of less than or equal to about 2 ml, preferably a volume of about 1 ml or less. Preferred formulations are ready-to-use liquid pharmaceutical compositions comprising about 24 mg/mL to about 120 mg/mL ligelizumab, in an aqueous solution containing L-histidine/L-histidine hydrochloride monohydrate as buffer agents, trehalose dehydrate as a stabilizer/tonicity adjuster, and polysorbate 20 as a surfactant.

As used herein, the phrase “container having a sufficient amount of the IgE antagonist to allow delivery of [a designated dose]” is used to mean that a given container (e.g., vial, pen, syringe) has disposed therein a volume of an IgE antagonist (e.g., as part of a pharmaceutical composition) that can be used to provide a desired dose. As an example, if a desired dose is 240 mg, then a clinician may use 2 mL from a container that contains an IgE antibody formulation with a concentration of 120 mg/mL, 1 mL from a container that contains an IgE antibody formulation with a concentration of 240 mg/mL, 0.5 mL from a container contains an IgE antibody formulation with a concentration of 480 mg/ml, etc. In each such case, these containers have a sufficient amount of the IgE antagonist to allow delivery of the desired 240 mg dose.

In some embodiments of the disclosed uses, methods, and kits, the dose of the IgE antibody (e.g., ligelizumab) or an antigen binding fragment thereof is about 240 mg, the IgE antibody (e.g., ligelizumab) or an antigen binding fragment thereof is comprised in a liquid pharmaceutical formulation at a concentration of 120 mg/ml, and 2 ml of the pharmaceutical formulation is disposed within two pre-filled syringes (PFS), injection pens, or autoinjectors, each having 1 ml of the pharmaceutical formulation. In this case, the patient receives two injections of 1 ml each, for a total dose of 240 mg, during each administration. In some embodiments, the dose of the IgE antibody (e.g., ligelizumab) or an antigen binding fragment thereof is about 240 mg, the IgE antibody (e.g., ligelizumab) or an antigen binding fragment thereof is comprised in a liquid pharmaceutical formulation at a concentration of 120 mg/ml, and 2 ml of the pharmaceutical formulation is disposed within an autoinjector or PFS. In this case, the patient receives one injection of 2 ml, for a total dose of 240 mg, during each administration. In methods employing one injection of 2 ml (e.g., via a single PFS or autoinjector) (i.e., a “single-dose preparation”), the drug exposure (AUC) and maximal concentration (Cₘₐₓ) is equivalent (similar to, i.e., within the range of acceptable variation according to US FDA standards) to methods employing two injections of 1 ml (e.g., via two PFSs or two AIs) (i.e., a “multiple-dose preparation”).

Disclosed herein are methods of treating chronic spontaneous urticaria (CSU), comprising subcutaneously (SC) administering to a patient in need thereof a dose of about 24 mg-about 240 mg of an IgE antibody (e.g., ligelizumab) or an antigen binding fragment thereof, weekly during week 0 and thereafter SC at a dose of about 24 mg-about 240 mg: a) monthly (every 4 weeks), beginning during week 4. Also disclosed herein is an IgE antibody (e.g. ligelizumab) or an antigen binding fragment thereof, for use in treating CSU, comprising subcutaneously (SC) administering to a patient in need thereof a dose of about 24 mg-about 240 mg of the IgE antibody or an antigen binding fragment thereof, weekly during week 0 and thereafter SC at a dose of about 24 mg-about 240 mg monthly (every 4 weeks), beginning during week 4. Alternatively, disclosed herein is an IgE antibody (e.g. ligelizumab) or an antigen binding fragment thereof, for use in the manufacture of a medicament for treating CSU, comprising subcutaneously (SC) administering to a patient in need thereof a dose of about 24 mg-about 240 mg of the IgE antibody or an antigen binding fragment thereof, weekly during weeks 0 and thereafter SC at a dose of about 24 mg-about 240 mg monthly (every 4 weeks), beginning during week 4.

Disclosed herein are methods of treating chronic spontaneous urticaria (CSU), comprising subcutaneously (SC) administering to a patient in need thereof a dose of about 24 mg-about 240 mg of an IgE antibody (e.g., ligelizumab) or an antigen binding fragment thereof, weekly during week 0 and thereafter SC at a dose of about 24 mg-about 240 mg: a) monthly (every 4 weeks), beginning during week 4, wherein the IgE antibody or antigen binding fragment thereof comprises an immunoglobulin V_{H} domain comprising the amino acid sequence set forth as SEQ ID NO:2 and an immunoglobulin V_{L} domain comprising the amino acid sequence set forth as SEQ ID NO:1.

Also disclosed herein is an IgE antibody (e.g. ligelizumab) or an antigen binding fragment thereof, for use in treating CSU, comprising subcutaneously (SC) administering to a patient in need thereof a dose of about 24 mg-about 240 mg of the IgE antibody or an antigen binding fragment thereof, weekly during week 0 and thereafter SC at a dose of about 24 mg-about 240 mg monthly (every 4 weeks), beginning during week 4, wherein the IgE antibody or antigen binding fragment thereof comprises an immunoglobulin VH domain comprising the amino acid sequence set forth as SEQ ID NO:2 and an immunoglobulin VL domain comprising the amino acid sequence set forth as SEQ ID NO:1.

Also disclosed herein is an IgE antibody (e.g. ligelizumab) or an antigen binding fragment thereof, for use in treating CSU, comprising subcutaneously (SC) administering to a patient in need thereof a dose of about 24 mg-about 240 mg of the IgE antibody or an antigen binding fragment thereof, weekly during week 0 and thereafter SC at a dose of about 24 mg-about 240 mg monthly (every 4 weeks), beginning during week 4, wherein the ligelizumab antibody comprises a variable light chain region comprising CDRL1, CDRL2, and CDRL3 and a variable heavy chain region comprising CDRH1, CDRH2, and CDRH3, wherein CDRL1 consists of SEQ ID NO:3, CDRL2 consists of SEQ ID NO:4, CDRL3 consists of SEQ ID NO:5, CDRH1 consists of SEQ ID NO:6, CDRH2 consists of SEQ ID NO:7, and CDRH3 consists of SEQ ID NO:8, wherein the antibody binds specifically to IgE.

In preferred embodiments of the disclosed methods, uses and kits, the dose of the IgE antibody or antigen binding fragment is about 24 mg or about 240 mg.

In preferred embodiments of the disclosed methods, uses and kits, the IgE antibody or antigen binding fragment thereof is administered SC at a dose of about 24 mg weekly during week 0 and thereafter SC at a dose of about 24 mg every four weeks, beginning during week 4.

In other preferred embodiments of the disclosed methods, uses and kits, the IgE antibody or antigen binding fragment thereof is administered SC at a dose of about 72 mg weekly during week 0 and thereafter SC at a dose of about 72 mg every four weeks, beginning during week 4.

In other preferred embodiments of the disclosed methods, uses and kits, the IgE antibody or antigen binding fragment thereof is administered SC at a dose of about 120 mg weekly during week 0 and thereafter SC at a dose of about 120 mg every four weeks, beginning during week 4.

In preferred embodiments of the disclosed methods, uses and kits, the patient achieves a sustained response after one year of treatment as measured by complete hives response (Hives Severity Score [HSS7]), UAS7 and Dermatology Life Quality Index (DLQI).

In preferred embodiments of the disclosed methods, uses and kits, prior to treatment with the IgE antibody or antigen binding fragment, the patient has been previously treated with a systemic agent for CSU.

In preferred embodiments of the disclosed methods, uses and kits, the systemic agent is selected from the group consisting of H1-antihistamines (H1-AH), H2-AH, and a leukotriene receptor antagonist (LTRA) and combinations thereof.

In some embodiments of the disclosed methods, uses and kits, prior to treatment with the IgE antibody or antigen binding fragment, the patient has not been previously treated with a systemic agent or a topical treatment for CSU.

In preferred embodiments of the disclosed methods, uses and kits, the dose of the IgE antibody or antigen binding fragment is about 24 mg. In other preferred embodiments of the disclosed methods, uses and kits, the dose of the IGE antibody or antigen binding fragment is about 72 mg. In other preferred embodiments of the disclosed methods, uses and kits, the dose of the IGE antibody or antigen binding fragment is about 120 mg. In other preferred embodiments of the disclosed methods, uses and kits, the dose of the IGE antibody or antigen binding fragment is about 240 mg.

In preferred embodiments of the disclosed methods, uses and kits, the patient has moderate to severe CSU.

In preferred embodiments of the disclosed methods, uses and kits, the patient is an adult. In some embodiments of the disclosed methods, uses and kits, the patient is an adolescent.

In preferred embodiments of the disclosed methods, uses and kits, the IgE antibody or antigen binding fragment is disposed in a pharmaceutical formulation, wherein said pharmaceutical formulation further comprises a buffer and a stabilizer. In some embodiments of the disclosed methods, uses and kits, the pharmaceutical formulation is in liquid form (ready-to-use). In some embodiments of the disclosed methods, uses and kits, the pharmaceutical formulation is in lyophilized form. In some embodiments of the disclosed methods, uses and kits, pharmaceutical formulation is disposed within pre-filled syringes, vials, injection pens, or autoinjectors.

In preferred embodiments of the disclosed methods, uses and kits, the dose of the IgE antibody or antigen binding fragment is about 24 mg, 72 mg, 120 mg, or 240 mg, the pharmaceutical formulation is disposed within means for administering selected from the group consisting of a pre-filled syringe, an injection pen, and an autoinjector, and said means is disposed within a kit, and the kit further comprises instructions for use.

In preferred embodiments of the disclosed methods, uses and kits, the dose of the IgE antibody or antigen binding fragment is about 24 mg, 72 mg, or 120 mg, the pharmaceutical formulation is disposed within an autoinjector or a pre-filled syringe, and the autoinjector or pre-filled syringe is disposed within a kit, and the kit further comprises instructions for use.

In preferred embodiments of the disclosed methods, uses and kits, the dose of the IgE antibody or antigen binding fragment is about 240 mg, the pharmaceutical formulation is disposed within autoinjectors or pre-filled syringes, the autoinjectors or pre-filled syringes are disposed within a kit, and the kit further comprises instructions for use.

In preferred embodiments of the disclosed methods, uses and kits, the dose is 240 mg, which is administered as a single subcutaneous administration in a total volume of 2 ml from a formulation comprising 120 mg/ml of the IgE antibody or antigen binding fragment, wherein the pharmacological exposure of the patient to the IgE antibody or antigen binding fragment is equivalent to the pharmacological exposure of the patient to the IgE antibody or antigen binding fragment using two separate subcutaneous administrations of a total volume of 1 ml each of the same formulation.

In preferred embodiments of the disclosed methods, uses and kits, the dose is 240 mg, which is administered as two separate subcutaneous administrations in a volume of 1 ml each from a formulation comprising 120 mg/ml of the IgE antibody or antigen binding fragment.

In preferred embodiments of the disclosed methods, uses and kits, prior to treatment with the IgE antibody or antigen binding fragment, the patient has a UAS7 score 16.

In preferred embodiments of the disclosed methods, uses and kits, prior to treatment with the IgE antibody or antigen binding fragment, the patient has an HSS7≥8.

In preferred embodiments of the disclosed methods, uses and kits, the patient achieves a HSS7 score of 0 by week 12.

In preferred embodiments of the disclosed methods, uses and kits, the patient achieves an UAS7 score of 0 by week 12 of treatment.

In preferred embodiments of the disclosure, the IgE antibody or antigen-binding fragment thereof is a monoclonal antibody.

In preferred embodiments of the disclosure, the IgE antibody or antigen-binding fragment thereof is a human or humanized antibody.

In preferred embodiments of the disclosure, the IgE antibody or antigen-binding fragment thereof is a human antibody.

In preferred embodiments of the disclosed methods, uses and kits, the IgE antibody or antigen binding fragment is a human monoclonal antibody.

In preferred embodiments of the disclosed methods, uses and kits, the IgE antibody or antigen binding fragment has a Tₘₐₓ of about 2-14 days.

In preferred embodiments of the disclosed methods, uses and kits, the IgE antibody or antigen binding fragment has an absolute bioavailability of about 47-100%.

In preferred embodiments of the disclosure, the IgE antibody or antigen-binding fragment thereof is ligelizumab.

### Kits

The disclosure also encompasses kits for treating CSU. Such kits comprise an IgE antagonist, e.g., IgE binding molecule (e.g., IgE antibody or antigen-binding fragment thereof, e.g., ligelizumab) or IgE receptor binding molecule (e.g., IgE antibody or antigen-binding fragment thereof) (e.g., in liquid or lyophilized form) or a pharmaceutical composition comprising the IgE antagonist (described supra). Additionally, such kits may comprise means for administering the IgE antagonist (e.g., an autoinjector, a syringe and vial, a prefilled syringe, a prefilled pen) and instructions for use. These kits may contain additional therapeutic CSU agents (described supra) for treating CSU, e.g., for delivery in combination with the enclosed IgE antagonist, e.g., IgE binding molecule, e.g., IgE antibody, e.g., ligelizumab. Such kits may also comprise instructions for administration of the IgE antagonist (e.g., IgE antibody, e.g., ligelizumab) to treat the CSU patient. Such instructions may provide the dose (e.g., 10 mg/kg, 24 mg, 72 mg, 120 mg, 240 mg), route of administration (e.g., IV, SC), and dosing regimen (e.g., weekly, monthly, weekly and then monthly, weekly and then every other week, etc.) for use with the enclosed IgE antagonist, e.g., IgE binding molecule, e.g., IgE antibody, e.g., ligelizumab.

The phrase “means for administering” is used to indicate any available implement for systemically administering a drug to a patient, including, but not limited to, a pre-filled syringe, a vial and syringe, an injection pen, an autoinjector, an IV drip and bag, a pump, etc. With such items, a patient may self-administer the drug (i.e., administer the drug without the assistance of a physician) or a medical practitioner may administer the drug. In some embodiments, a total dose of 240 mg is to be delivered in a total volume of 2 ml that is disposed in two PFSs or autoinjectors, each PFS or autoinjector containing a volume of 1 ml having 120 mg/ml of the IgE antibody, e.g., ligelizumab. In this case, the patient receives two 1 ml injections (a multi-dose preparation). In preferred embodiments, a total dose of 240 mg is to be delivered in a total volume of 2 ml having 120 mg/ml of the IgE antibody, e.g., ligelizumab, which is disposed in a single PFS or autoinjector. In this case, the patient receives one 2 ml injection (a single dose preparation).

Disclosed herein are kits for use treating a patient having CSU, comprising an IgE antagonist (e.g., IgE binding molecule, e.g., IgE antibody or antigen-binding fragment thereof, e.g., ligelizumab) and means for administering the IgE antagonist to the CSU patient. In some embodiments, the kit further comprises instructions for administration of the IgE antagonist, wherein the instructions indicate that the IgE antagonist (e.g., IgE binding molecule, e.g., IgE antibody or antigen-binding fragment thereof, e.g., ligelizumab) is to be administered to the patient SC at about 24 mg-about 240 mg (e.g., about 24 mg, or about 240 mg) at week 0 and thereafter SC at about 24 mg-about 240 mg (e.g., about 24 mg, about 240 mg) monthly (every 4 weeks) thereafter beginning during week 4.

### General

In the most preferred embodiments of the disclosed uses, methods and kits, the IgE antagonist is an anti-IgE antibody or antigen-binding fragment thereof.

In most preferred embodiments of the disclosed methods, kits, or uses, the IgE antibody or antigen-binding fragment thereof is a monoclonal antibody. In most preferred embodiments of the disclosed methods, kits, or uses the IgE antibody or antigen-binding fragment thereof is a human or humanized antibody, preferably a human antibody. In most preferred embodiments of the disclosed methods, kits, or uses, the antibody or antigen-binding fragment thereof is ligelizumab.

In most preferred embodiments of the disclosed methods, kits, or uses, the antibody or antigen-binding fragment thereof is ligelizumab, the dose size is flat (also referred to as a “fixed” dose, which differs from weight-based or body surface area-based dosing), the dose is 24 mg, 72 mg, 120 mg or 240 mg, the route of administration is SC, and the regimen is administration at week 0 and then every four week, beginning during week 4. In preferred embodiments of the invention, the antibody or antigen-binding fragment thereof is given to the patient as a flat dose.

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural references unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference. The following Examples are presented in order to more fully illustrate the preferred embodiments of the disclosure. These examples should in no way be construed as limiting the scope of the disclosed subject matter, as defined by the appended claims.

### EXAMPLES

### Example 1: Efficacy and Safety Data in CSU Adult Patients Who Remain Symptomatic Despite Treatment with Standard of Care

Clinical evidence of the effects of an anti-IgE antibody, Xolair® (omalizumab), supports the potential of an anti-IgE antibody as an effective therapy for patients with CSU. Like ligelizumab, omalizumab is a recombinant fully human anti-IgE monoclonal antibody for the treatment of asthma and CSU. Ligelizumab binds with higher affinity to human IgE than omalizumab.

A phase 2b study (CQGE031C2201) was conducted as a multicenter, randomized, double-blind, placebo and active-controlled Phase 2b dose-finding study of ligelizumab (QGE031) as add-on therapy to investigate the efficacy and safety in patients with Chronic Spontaneous Urticaria (CSU). The study was a, randomized, double blind, active and placebo controlled, parallel group study to establish a dose-response relationship of ligelizumab, and evaluate its efficacy and safety compared to placebo and omalizumab.

| | |
|---|---|
| Purpose and | The purpose of this study was to collect efficacy and safety |
| rationale | data in CSU adult patients who remain symptomatic despite |
| | treatment with standard of care. |
| Primary | To establish the dose-response relationship of QGE031 with |
| Objective(s) | respect to achievement of complete hives response at Week 12 in |
| | patients with CSU when added to standard of care treatment |
| Secondary | To evaluate the efficacy of QGE031 (based on the selected |
| Objectives | dose-response model) compared to omalizumab with respect to |
| | achievement of complete hives response at Week 12 in patients |
| | with CSU when added to standard of care treatment. |
| | To evaluate the efficacy of various QGE031 doses compared to |
| | omalizumab with respect to achievement of complete hives |
| | response at Week 20. |
| | To evaluate the efficacy of various QGE031 doses versus |
| | placebo and omalizumab in patients with CSU, in terms of: |
| | Change from Baseline in Hives Severity Score (HSS7) at Week 12 and 20 |
| | Change from Baseline in Itch Severity Score (ISS7) at Week 12 and 20 |
| | Change from Baseline in Urticaria Activity Score (UAS7) at Week 12 and 20 |
| | To evaluate the safety |
| Study design | This was a Phase 2b dose-finding, multicenter, randomized, |
| | double blind, active and placebo controlled, parallel group study to |
| | establish a dose-response relationship of QGE031, and evaluate its |
| | efficacy and safety compared to placebo and omalizumab |
| | administered subcutaneously as an add-on therapy for the treatment |
| | of adult patients diagnosed with refractory CSU who remain |
| | symptomatic despite standard of care treatment. |
| Population | The study population consisted of approximately 360 adult |
| | males and females aged ≥18 and ≤75 years who were diagnosed |
| | with refractory CSU and who remained symptomatic despite |
| | standard of care treatment. |
| Inclusion | 1. Signed written informed consent before any assessment were performed. |
| criteria | 2. Male and female adult patients aged ≥18 to ≤75 years. |
| | 3. Diagnosis of CSU (with or without urticarial dermographism |
| | when testing for dermographism) refractory to standard of care |
| | treatment at the time of randomization, as defined by all of the |
| | following: The presence of itch and hives for ≥6 consecutive weeks at any |
| | time prior to enrollment despite current use of standard of care |
| | treatment during this time period |
| | UAS7 score ≥16 and HSS7 ≥8 during 7 days prior to |
| | randomization |
| | In-clinic UAS ≥4 on at least one of the screening visit days |
| | Patients must have been on standard of care for treatment of |
| | CSU for at least the 3 consecutive days immediately prior to the |
| | screening visit and must have documented current use on the |
| | day of the initial screening visit |
| | CSU diagnosis for ≥6 months |
| | 4. Willing and able to complete a daily symptom eDiary for the |
| | duration of the study and adhere to the study visit schedules. |
| Key Exclusion | Clearly defined underlying etiology for chronic urticarias other |
| criteria | than CSU. This includes the following: |
| | Inducible urticaria: urticaria factitia, cold-, heat-, solar-, |
| | pressure-, delayed pressure-, aquagenic-, cholinergic-, or |
| | contact-urticaria |
| | Diseases with possible symptoms of urticaria or angioedema |
| | such as urticarial vasculitis, erythema multiforme, cutaneous |
| | mastocytosis (urticaria pigmentosa), and hereditary or acquired |
| | angioedema (e.g., due to C1 inhibitor deficiency) |
| | Any other skin disease associated with chronic itching that |
| | might confound the study evaluations and results (e.g., atopic |
| | dermatitis, bullous pemphigoid, dermatitis herpetiformis, senile |
| | pruritus etc.) |
| | Previous exposure to omalizumab or QGE031 |
| | History of anaphylaxis |
| Investigational | QGE031: 24 mg, 72 mg or 240 mg s.c. q4w and 120 mg s.c. |
| and reference therapy | single dose |
| | Placebo: s.c. q4w (see Table 3) |
| | Omalizumab: s.c. q4w |
| Efficacy | Weekly Hives Severity Score (HSS7) |
| assessments | Weekly Itch Severity Score (ISS7) |
| | Weekly Urticaria Activity Score (UAS7) |
| Safety | Safety evaluations include adverse |
| assessments | events, laboratory values, vital signs and ECG |
| Other | Patient reported outcome |
| assessments | PK/PD/PG/Biomarkers |
| Data analysis | The main purpose of this study was to characterize the dose |
| | response relationship among QGE031 doses and placebo with |
| | respect to achievement of complete hives response after 12 weeks |
| | of treatment, and select an appropriate dose (or range of doses) to |
| | use in Phase 3 studies, which is likely to be superior to |
| | omalizumab. The consecutive steps are therefore (1) to confirm an |
| | overall dose-response signal, (2) to estimate the minimal QGE031 |
| | dose that shows a relevantly superior effect over omalizumab based |
| | on the selected dose-response model. The MCP-Mod methodology |
| | was used to address these goals. |

Study Objectives

Primary Objective(s) To establish the dose-response relationship of QGE031 with respect to achievement of complete hives response at Week 12 in patients with CSU when added to H1-AH alone or in combination with H2-AH and/or a LTRA.

Complete hives response is defined as a HSS7 score of 0. Analogously, complete itch response and complete UAS7 response are defined as an ISS7 and UAS7 score of 0, respectively.

Secondary Objectives
To evaluate the efficacy of QGE031 (based on the selected dose-response model) compared to omalizumab 300 mg with respect to achievement of complete hives response at Week 12 in patients with CSU when added to H1-AH alone or in combination with H2-AH and/or a LTRA.
To evaluate the efficacy of individual QGE031 doses of 24 mg, 72 mg and 240 mg s.c. compared to omalizumab 300 mg with respect to achievement of complete hives response at Week 20 in patients with CSU when added to H1-AH alone or in combination with H2-AH and/or a LTRA.
To evaluate the efficacy of QGE031 doses of 24 mg, 72 mg and 240 mg s.c. versus placebo and omalizumab 300 mg in patients with CSU, in terms of:
Change from Baseline in Hives Severity Score (HSS7) at Week 12 and 20
Change from Baseline in Itch Severity Score (ISS7) at Week 12 and 20
Change from Baseline in Urticaria Activity Score (UAS7) at Week 12 and 20
To evaluate the safety (including immunogenicity) and tolerability of QGE031 (doses of 24 mg, 72 mg and 240 mg s.c. every 4 weeks) versus placebo and omalizumab 300 mg in patients with CSU particularly in regards to ECG, adverse events, vital signs and clinical laboratory evaluation during 20 weeks of treatment and 24 weeks of follow-up.

Exploratory Objectives
To explore the efficacy of QGE031 (doses of 24 mg, 72 mg and 240 mg s.c. every 4 weeks) versus placebo and omalizumab 300 mg in terms of:
Achievement of complete UAS7 response at Week 12 and 20
Achievement of complete itch response at Week 12 and 20
Achievement of Urticaria Activity Score (UAS7)≤6 at Week 12 and 20
Profile of change from baseline of HSS7, ISS7 and UAS7 over time
Time to onset of clinical effect (e.g., HSS7, UAS7, ISS7)
Correlation of clinical response with pharmacodynamic parameters related to the IgE pathway (e.g., total IgE, basophil IgE expression)
Impact of CU Index status (+ or −) on treatment effect
Sleep interference and daily activity interference collected on Urticaria Patient Daily Diary (UPDD)
Use of rescue medication
Occurrence of and actions/treatments for angioedema collected on UPDD
Duration of response after withdrawal of drug
Number of calls to doctor, nurse or nurse practitioner
Utility of the in-clinic assessment of angioedema
Change from baseline to Week 12 (and 20) in the following PRO assessments:
Dermatology Life Quality Index (DLQI) total score
Angioedema Activity Score (AAS)
e.g. weekly number of angioedema episodes/days
Work Productivity and Activity Impairment (WPAI)-CU
To assess the pharmacokinetics (PK) of QGE031
To perform exploratory pharmacogenetic assessments to examine whether individual genetic variation in genes relating to drug metabolism, CSU, and the drug target pathway confer differential response to QGE031
To assess the potential utility of the biomarkers in terms of QGE031 efficacy and safety correlations

Study Design

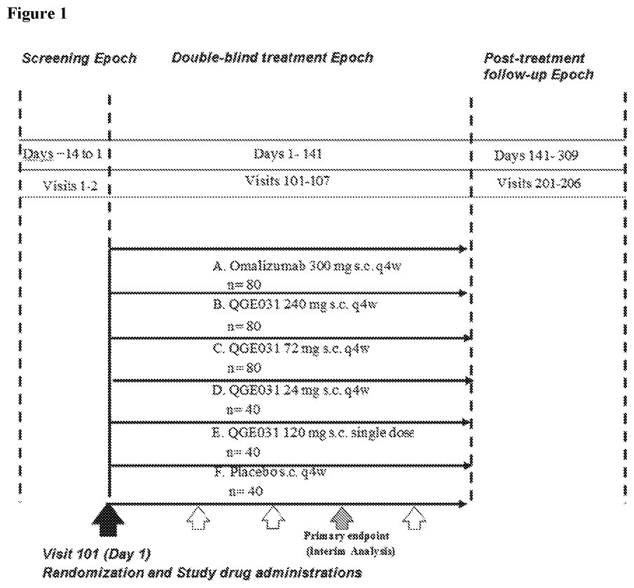

This was a Phase 2b dose-finding, multicenter, randomized, double-blind, active and placebo-controlled, parallel-group study to establish a dose-response relationship of QGE031, and evaluate its efficacy and safety compared to placebo and omalizumab administered subcutaneously as an add-on therapy for the treatment of adult patients diagnosed with refractory CSU who remained symptomatic despite H1-AH at approved or increased doses alone or in combination with H2-AH and/or a leukotriene receptor antagonist (LTRA). The study consisted of three distinct epochs over 46 weeks, as outlined below (see also FIG. 1):
Screening epoch, Day −14 to Day 1: Duration of up to 2 weeks in which patients who have given informed consent were assessed for study eligibility.
Treatment epoch, Day 1 to Day 141 (20 weeks): Double-blind treatment epoch during which patients were seen in the clinic every 4 weeks.
Post-treatment follow-up epoch, Day 141 to Day 309 (24 weeks): Follow-up epoch consists of 6 visits (every four weeks) with the final visit occurring up to 24 weeks after the last treatment visit or when patients relapse within the follow-up epoch from Week 32 onwards.

Screening Epoch

Patients had up to a 2 week screening epoch to establish eligibility for the study. Patients were required to attend two visits during the screening epoch: at Day −14 and Day −7. Only in exceptional circumstances, when information concerning eligibility was outstanding (e.g., pending laboratory data), was an extended screening epoch permitted.

For certain Inclusion/Exclusion criteria, rescreening was allowed for patients who failed initial screening. Only one rescreening was allowed. If a patient rescreened for the study, the patient signed a new informed consent and he/she was issued a new patient number. Informed consent for a rescreened patient was obtained prior to performing any study-related assessments or collecting any data for the Screening visit.

Double-Blind Treatment Epoch

On Day 1, eligible patients were randomly assigned to receive QGE031 24 mg, 72 mg or 240 mg, or omalizumab 300 mg or placebo s.c. q4w or to QGE 120 mg single dose s.c. injection (to maintain the blind, subsequent placebo injections will be given) during the 20-week double-blind treatment epoch. It was planned to allocate approximately 80 patients each to the QGE031 240 mg q4w, 72 mg q4w and to the omalizumab 300 mg q4w arms. Approximately 40 patients each were allocated to the QGE031 24 mg q4w, placebo q4w and QGE031 120 mg single dose arms. Patients were expected to attend all site visits based on the assessment schedule (Table 2).

TABLE 2
Assessment schedule

| | | Epoch | | | | | | | | | | | | | | |
|---|---|---|---|---|---|---|---|---|---|---|---|---|---|---|---|---|
| | | Screening | | Double-blind Treatment | | | | | | | Post-treatment Follow-Up | | | | | |
| | | Visit | | | | | | | | | | | | | | |
| | | | | | | | | | | 107 | | | | | | 206 |
| | | | | | | | | | | or | | | | | | or |
| | | 1 | 2 | 101 | 102 | 103 | 104 | 105 | 106 | PSW | 201 | 202 | 203 | 204 | 205 | PSW |
| | | Day | | | | | | | | | | | | | | |
| | | −14 | −7 | 1 | 8 | 29 | 57 | 85 | 113 | 141 | 169 | 197 | 225 | 253 | 281 | 309 |
| | | week (R = randmization week) | | | | | | | | | | | | | | |
| | | −2 | −1 | R | 1 | 4 | 8 | 12 | 16 | 20 | 24 | 28 | 32 | 36 | 40 | 44 |
| Obtain Informed Consent | | D | | | | | | | | | | | | | | |
| Pharmacogenetics and/or | | D | | | | | | | | | | | | | | |
| Additional Biomarker | | | | | | | | | | | | | | | | |
| Informed Consent | | | | | | | | | | | | | | | | |
| Inclusion/exclusion criteria | | D | D | D | | | | | | | | | | | | |
| Demographic data | | D | | | | | | | | | | | | | | |
| Relevant medical history | | D | | | | | | | | | | | | | | |
| Family malignancy history | | | D | | | | | | | | | | | | | |
| Prior urticaria treatment | | D | | | | | | | | | | | | | | |
| Prior and Concomitant | | D | D | D | D | D | D | D | D | D | D | D | D | D | D | D |
| medication usage | | | | | | | | | | | | | | | | |
| Surgery and procedures | | D | D | D | D | D | D | D | D | D | D | D | D | D | D | D |
| Re-screening (re-screening | | D | | | | | | | | | | | | | | |
| CRF should be recorded if | | | | | | | | | | | | | | | | |
| the patient is re-screened) | | | | | | | | | | | | | | | | |
| Rescue medication usage | | D | | D | | D | D | D | D | D | D | D | D | D | D | |
| Dispense H1-AH rescue | | S | | S | | S | S | S | S | S | S | S | S | S | S | |
| medication | | | | | | | | | | | | | | | | |
| Dispense epinephrine | | | | S | | | | | | | | | | | | |
| Study drug/placebo | | | | D | | D | D | D | D | | | | | | | |
| administration | | | | | | | | | | | | | | | | |
| Randomization | | | | DS | | | | | | | | | | | | |
| Contact IRT | | DS | | DS | | DS | DS | DS | DS | DS | DS | | | | | DS |
| In-clinic assessment of UAS | | D | D | D | D | D | D | D | D | D | D | D | D | D | D | D |
| In-clinic assessment of | | D | | D | | D | D | D | D | D | D | D | D | D | D | D |
| angioedema (Investigator | | | | | | | | | | | | | | | | |
| Global Assessment) | | | | | | | | | | | | | | | | |
| ePROs should be completed | | | | | | | | | | | | | | | | |
| prior to any other assessment | | | | | | | | | | | | | | | | |
| Dispense Patients' eDiary | | S | | | | | | | | | | | | | | |
| Patients' eDiary (download | | | DS | DS | DS | DS | DS | DS | DS | DS | DS | DS | DS | DS | DS | DS |
| and review) includes the | | | | | | | | | | | | | | | | |
| urticaria patient daily diary | | | | | | | | | | | | | | | | |
| (UPDD) and AAS (Angioedema | | | | | | | | | | | | | | | | |
| activity score). | | | | | | | | | | | | | | | | |
| DLQI (Dermatology Life | | | | DS | | DS | | DS | | DS | | DS | | DS | | DS |
| Quality Index) | | | | | | | | | | | | | | | | |
| WPAI (Work productivity and | | | | DS | | DS | | DS | | DS | | DS | | DS | | DS |
| activity impairment) | | | | | | | | | | | | | | | | |
| Laboratory tests | | | | | | | | | | | | | | | | |
| Stool ova and parasite | | D | | | | | | | | | | | | | | D |
| evaluation If diarrhea develops | | | | | | | | | | | | | | | | |
| at any time prior to the last | | | | | | | | | | | | | | | | |
| study drug administration, | | | | | | | | | | | | | | | | |
| additional assessments for | | | | | | | | | | | | | | | | |
| parasitic conditions will | | | | | | | | | | | | | | | | |
| be performed | | | | | | | | | | | | | | | | |
| Blood | Hematology (plus INR) | DS | | DS | DS | DS | DS | DS | DS | DS | DS | DS | D | DS | DS | DS |
| sampling | Clinical chemistry | DS | | DS | DS | DS | DS | DS | DS | DS | DS | DS | DS | DS | DS | DS |
| | including hs-CRP | | | | | | | | | | | | | | | |
| | and bilirubin (all | | | | | | | | | | | | | | | |
| | indicated visits), | | | | | | | | | | | | | | | |
| | hCG (Visit 1 only) | | | | | | | | | | | | | | | |
| | PK/PD: QGE031 | | | DS | DS | DS | DS | DS | DS | DS | DS | DS | DS | DS | DS | DS |
| | total IgE | | | | | | | | | | | | | | | |
| | Anti-Drug/QGE031 | | | DS | | DS | | DS | | DS | | DS | | | | DS |
| | antibodies (ADA) | | | | | | | | | | | | | | | |
| | Chronic urticaria | | | DS | | | | | | | | | | | | |
| | (CU) index panel | | | | | | | | | | | | | | | |
| | (CU index, thyroid | | | | | | | | | | | | | | | |
| | peroxidase IgG, | | | | | | | | | | | | | | | |
| | thyroglobulin IgG,) | | | | | | | | | | | | | | | |
| | Pharmacogenetics | | | DS | | | | | | | | | | | | |
| | (Sample collected | | | | | | | | | | | | | | | |
| | only if patient | | | | | | | | | | | | | | | |
| | has signed | | | | | | | | | | | | | | | |
| | Pharmacogenetic | | | | | | | | | | | | | | | |
| | Informed Consent) | | | | | | | | | | | | | | | |
| | IgE-autoantibodies | | | DS | | | | | | | | | | | | D |
| | (Marker of | | | | | | | | | | | | | | | S |
| | hypersensitivity) | | | | | | | | | | | | | | | |
| | Platelet-activating | | | DS | | | | | | | | | | | | |
| | factor-acetylhydrolase | | | | | | | | | | | | | | | |
| | (PAF-AH/ Lp-PLA2) | | | | | | | | | | | | | | | |
| | (Marker of | | | | | | | | | | | | | | | |
| | hypersensitivity) | | | | | | | | | | | | | | | |
| | Total tryptase | | | DS | DS | DS | | | | DS | | | | | | DS |
| | Additional biomarkers | | | DS | DS | DS | | DS | | DS | DS | DS | DS | | | DS |
| | (Sample collected only | | | | | | | | | | | | | | | |
| | if patient has signed | | | | | | | | | | | | | | | |
| | Additional Biomarker | | | | | | | | | | | | | | | |
| | Informed Consent) | | | | | | | | | | | | | | | |
| Urine | Urinalysis (local) | | D | | | | | | | D | | | | | | D |
| sampling | When a dipstick | | | | | | | | | | | | | | | |
| | evaluation is abnormal, | | | | | | | | | | | | | | | |
| | e.g. positive for WBC | | | | | | | | | | | | | | | |
| | and/or blood, a | | | | | | | | | | | | | | | |
| | microscopic examination | | | | | | | | | | | | | | | |
| | including RBC and WBC | | | | | | | | | | | | | | | |
| | should be done by | | | | | | | | | | | | | | | |
| | central lab | | | | | | | | | | | | | | | |
| | Urine Pregnancy | | | D | | D | D | D | D | D | D | D | D | | | D |
| | Test if positive to be | | | | | | | | | | | | | | | |
| | confirmed by serum test | | | | | | | | | | | | | | | |
| | should be done be | | | | | | | | | | | | | | | |
| | central lab | | | | | | | | | | | | | | | |
| | Urine mast cell | | | DS | DS | DS | | | | DS | | | | | | DS |
| | metabolites plus | | | | | | | | | | | | | | | |
| | Urine creatinine | | | | | | | | | | | | | | | |

| | | | | | | | | | | | | | | | |
|---|---|---|---|---|---|---|---|---|---|---|---|---|---|---|---|
| Safety | | | | | | | | | | | | | | | |
| Physical Exam | S | S | S | S | S | S | S | S | S | S | S | S | S | S | S |
| (Physical exam on Day −14 is | | | | | | | | | | | | | | | |
| comprehensive but subsequent | | | | | | | | | | | | | | | |
| physical exams maybe limited) | | | | | | | | | | | | | | | |
| Vital signs | D | | D | | D | D | D | D | D | D | D | D | D | D | D |
| Height and Weight (at visits | D | | | | | | | | D | | | | | | D |
| 107 and 206 only body weight | | | | | | | | | | | | | | | |
| will be measured) | | | | | | | | | | | | | | | |
| ECG (three ECG measurements | D | | | | | D | | | D | | | | | | D |
| to be taken at approximately 1 | | | | | | | | | | | | | | | |
| minute intervals at Visit 1 and 107, | | | | | | | | | | | | | | | |
| all other visits a single ECG | | | | | | | | | | | | | | | |
| measurement will be recorded) | | | | | | | | | | | | | | | |
| Adverse events | D | D | D | D | D | D | D | D | D | D | D | D | D | D | D |
| Liver safety monitoring | D | | D | D | D | D | D | D | D | D | D | D | | | D |
| Renal safety monitoring | D | | D | D | D | D | D | D | D | D | D | D | | | D |
| Others | | | | | | | | | | | | | | | |
| Screening epoch | | D | | | | | | | | | | | | | |
| completion form | | | | | | | | | | | | | | | |
| Double-blind-treatment | | | | | | | | | D | | | | | | |
| epoch completion | | | | | | | | | | | | | | | |
| Post-treatment follow-up | | | | | | | | | | | | | | | D |
| epoch completion | | | | | | | | | | | | | | | |

S = assessment to be recorded on source documentation only
D = assessment to be recorded on the CRF based on source documentation
DS = assessment to be loaded into the database from separate source documents, i.e. outside vendors
PSW = Premature Study Withdrawal: Patients who discontinue study treatment early will be expected to perform the Week 20 (Visit 107) assessments four weeks after their last dose. These patients will subsequently be expected to perform all follow-up evaluations (Visit 201-206). Patients who are within the follow-up epoch but withdraw from the study early will be expected to perform Visit 206 assessments.
Note:
All assessments should be performed pre-administration of study drug if applicable

The last dose of study drug during the treatment epoch was administered at the Day 113 (Week 16) study visit. As background medication, all patients in this study continued to receive H1-AH, either alone or in combination with H2-AH and/or LTRA. Patients remained on a stable treatment regimen throughout the study. The primary analysis was planned when all patients reached Week 12. End-of-treatment analysis was planned at Week 20 of the treatment period.

Post-Treatment Follow-Up Epoch

After the completion of the double-blind treatment epoch, patients entered a post-treatment follow-up epoch to allow for further characterization of the PK and PD of QGE031, collection of additional efficacy and safety data (e.g., relapse), and evaluation of the presence of anti-drug antibodies (ADAs). The follow-up epoch was 24 weeks with the last follow-up visit (Visit 206) corresponding to 28 weeks after the last treatment dose. No investigational treatment was given during the post-treatment follow-up epoch, however, patients were allowed to take their rescue medication. Patients were required to visit the study center every four weeks during post-treatment epoch.

Patients who did not discontinue investigational treatment and had a UAS7 score ≥12 at Visit 203 (Week 32, 16 weeks after last injection) or at the following visits (204 to 206) were also eligible to enter the extension study prior to completion of the follow-up epoch.

Patients were not allowed to enter the extension study prior Visit 203 due to insufficient wash-out of core study medication in particular for patients in the omalizumab arm.

Rescue medication (Loratadine, or Fexofenadine, or Cetirizine) were provided and used on an as-needed basis during the screening, treatment, and post-treatment follow-up epochs.

Rationale of Study Design

This randomized, double-blind, parallel-group, placebo- and active-controlled design supports the dose-range finding and assessment of efficacy as well as safety. The study was designed as a dose-range finding study with the aim to establish a dose-response relationship, and, based on the selected dose-response model, to identify a dose which has a benefit to patients with uncontrolled CSU versus omalizumab 300 mg, the only alternative anti-IgE medication that is currently marketed in many countries.

The target population for this study consisted of CSU patients who remain symptomatic despite treatment with H1-AH alone (at approved or increased doses), or in combination with H2-AH and/or a LTRA. These patients had a significant unmet medical need and represent the target population for QGE031. In the omalizumab development program, differences emerged between the FDA and EMA expectations with regard to the background medications (H1-AH vs combination of H1-AH, H2-AH and/or LTRA). No significant differences in efficacy were observed between the two populations in the omalizumab Phase 3 studies. Since the start of the omalizumab development program and the scientific advice given by FDA and EMA in 2009 and 2010, new medical data and scientific progress have led to an updated International Guideline on definition, classification, diagnosis and management of urticaria (Zuberbier T, Aberer W, Asero R, et al (2014) The EAACI/GA(2) LEN/EDF/WAO Guideline for the definition, classification, diagnosis, and management of urticaria: the 2013 revision and update. Allergy; 69(7):868-87). Evidence based recommendations by an international expert team led to a treatment algorithm that recommends for step 1 and 2 the use of non-sedating H1-AHs at approved, or increased doses (up to fourfold), respectively. Only as step 3 the addition of omalizumab, ciclosporin A, or montelukast is recommended.

Therefore, to reflect the most current treatment algorithm for CSU, this study allowed use of H1-AH alone (at approved doses as per local health authority guidance or up to fourfold increased doses) or in combination with H2-AH and/or LTRA as background medications. However, they may be separated in the Phase 3 program (pending confirmation with Health Authorities). To allow for comparison to the existing CSU data from the omalizumab program, the population was otherwise nearly identical to that studied in the omalizumab Phase 3 studies.

The use of placebo in this patient population was considered to be appropriate since patients will continue with their background therapy. They were also allowed to take H1-AH as rescue medication throughout the study. In addition, patients were allowed to enter an extension study (long-term safety study) where they received QGE031. Although the signs and symptoms of CSU are burdensome to patients, placebo trials have been safely and successfully conducted in this indication (Kaplan A, Ledford D, Ashby M, et al (2013) Omalizumab in patients with symptomatic chronic idiopathic/spontaneous urticaria despite standard combination therapy. J Allergy Clin Immunol; 132(1):101-9; Maurer M, Magerl M, Metz M, et al (2013) Revisions to the international guidelines on the diagnosis and therapy of chronic urticaria. J Dtsch Dermatol Ges.). Patients were allocated across the treatment arms according to the number needed to satisfy data requirements for optimal modeling.

Administration of placebo has been limited as far as possible while still allowing statistical comparison and maintenance of blinding to active versus placebo treatment. Double-dummy design was not possible due to the absence of a placebo that would completely match the characteristics of omalizumab hence increasing risk of un-blinding personnel and patients (e.g. potential for patients to detect differences in sensation between the injections delivered); it would also have increased burden/discomfort to patients in terms of total number of injections at each visit. Therefore, this study will use placebo to QGE031 arms only. As the volume administered for QGE031 and omalizumab will vary (e.g. QGE031 240 mg has a volume of 2.0 mL administered as 2 separate 1.0 mL injections, omalizumab 300 mg has a volume of 2.4 mL administered as 2 separate 1.2 mL injections), treatment arms will have differing injection volumes.

In addition, to maintain blinding, the study drug was administered by an unblinded person (e.g., study nurse or physician) that was independent from the site team performing study assessments.

The primary analysis was performed when all patients reached Week 12. End-of-treatment analysis was performed at Week 20 of the treatment period.

Rationale of Dose/Regimen, Route of Administration and Duration of Treatment

It is hypothesized that the greater potency of QGE031 to suppress IgE relative to omalizumab translates to greater benefit in the treatment of CSU. For a scientifically valid assessment, comparison with both placebo and omalizumab treatment groups was included for interpretation of the response to QGE031.

Rationale for Doses/Regimens

The dose-response model was derived from testing a 10-fold range of QGE031 doses starting at 24 mg q4w over 72 mg q4w to 240 mg q4w with placebo as the zero dose. The dose-response model was then be used to find a dose or dose range which adds the desired benefit over the active comparator.

QGE031 24, 72 and 240 mg was administered s.c q4w for 20 weeks, i.e. a total 5 administrations. Omalizumab 300 mg was administered s.c. q4w as the active comparator, and the control was placebo matched to the dosing regimen.

The rationale for selecting 120 mg as the dose for the single dose arm was to ensure a maximal or close to maximal effect in the first days post dose, but not so high as to have patients not relapse within the trial period. This arm provided blinded washout data that are critical to determine the level of drug in the serum associated with the return of itch and hive symptoms, from which alternative dosing intervals than q4w.

The rationale for the q4w doses/regimen was based on (a) allergen skin prick test tolerance data from studies QGE031A2103 with atopic volunteers and QGE031B2203 with asthma patients and (b) omalizumab efficacy data from Phase 3 studies with CSU patients.

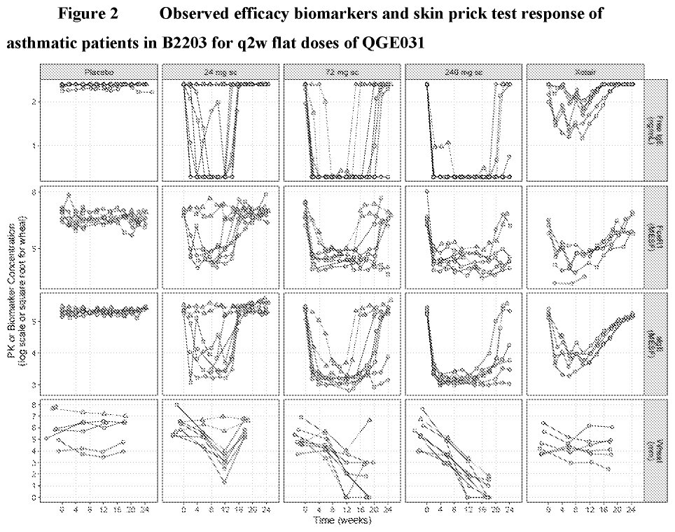

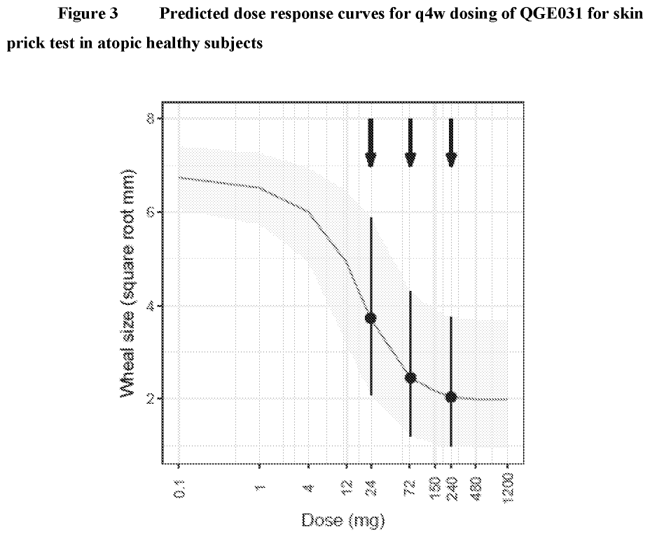

(a) Allergen Skin Prick Test Tolerance Data
The skin prick test was considered as a predictor of the urticarial response as it is a direct demonstration of the level of IgE pathway responsiveness in skin and the histamine mediated wheal response is shared between skin prick testing and urticaria. The clinical readout was based on the diameter of the wheals appearing in the skin prick test. FIG. 2 shows the reduction of free IgE in plasma and reduction of FSRI and surface IgE on peripheral basophils as well as reduction of the wheal component of the skin prick test in asthma patients in study QGE031B2203. For patients with moderate IgE levels both 72 mg and 240 mg lead to pronounced reduction of the biomarkers and efficacy in the skin prick test. Similar observations were made for the healthy volunteers in QGE031A2103 that used weight adjusted dosing.
The dosing regimen in QGE031A2103 and QGE031B2203 were every 2 weeks (q2w) such that the data was not directly informative on appropriate doses of a q4w regimen. Hence, using a model established on this data was used to project the dose response relationship for q4w dosing in a CSU population.
FIG. 3 shows the predicted dose response curves for QGE031 for the wheal component of the skin prick test. A 24 mg dose was predicted to achieve 50-70% of the maximal possible response. The range of responses for the middle 50% of the patient population spans from a very small to a high but not maximal response. A dose of 72 mg presumably was close to the transition between the linear and the saturated region of the dose response curve while 240 mg was anticipated to achieve maximal efficacy. Therefore, 24 mg represents a ‘sub optimal dose’ that was anticipated to be in the same range as omalizumab, rather than a minimally effective dose. Omalizumab 300 mg q4w is predicted to give a response slightly below that for QGE031 72 mg q4w.

(b) Omalizumab Efficacy Data from Phase 3 Studies in CSU
The Phase 3 clinical data of omalizumab was used as an alternative estimation of the clinical response for the selected QGE031 doses. Based on this data a PK/PD model was established linking the reduction in free IgE to the reduction in CSU symptom scores. Assuming different IgE binding affinity differences between QGE031 and omalizumab it was anticipated that 24 mg could achieve similar sub-optimal responses as 150 mg omalizumab. Only in the extreme case that the in vivo potency difference in CSU patients is similar to the in vitro binding affinity difference (50×), 24 mg might also be close to the maximal efficacy such that the concentration-response relationship has to be inferred from washout data. A dose of 240 mg QGE031 was predicted to achieve a much higher reduction in free IgE as compared to 300 mg omalizumab, which was expected to result in superior clinical efficacy. Note that the maximal effect level for symptom score reduction was estimated based on the omalizumab data which assumes that 300 mg omalizumab was already close to the maximal response. However, there is little data on the response for higher exposure, i.e. more pronounced IgE reduction, and therefore the model was not able to address the maximal response that might be observed with QGE031, a more potent drug.

In summary, either using the skin prick test data or projecting CSU efficacy data from omalizumab to QGE031 the doses 24 and 240 mg were predicted to result in suboptimal to maximal responses. The dose of 240 mg tested whether a higher free IgE reduction results in a higher efficacy as compared to 300 mg omalizumab. 72 mg is equally spaced on a log scale between these doses.

FIG. 3 shows the predicted dose response curves at week 20 based on simulations from a QGE031 PKPD model which had been fitted to the wheal component of skin prick test data from atopic but otherwise healthy subjects. The bands indicate the 25th and 75th percentiles representing variation between healthy subjects and the line shows the median. The units for the y-axis response are the square root of the sum of wheal sizes (in mm) of all allergen dilutions tested for each patient at each visit.

The rationale for fixed dosing:
The rationale for fixed rather than mg/kg dosing was to broaden (as much as is possible) the extent of exposure to QGE031 across the patients being enrolled, thereby enhancing the assessment of safety. The influence of bodyweight was, in any case, be investigated in the statistical and pharmacokinetic-pharmacodynamic modeling using data from this and other studies. (Wang D D, Zhang S, Zhao H, et al (2009) Fixed dosing versus body size-based dosing of monoclonal antibodies in adult clinical trials. J Clin Pharmacol; 49(9):1012-24) showed that the distributions of monoclonal antibody area under the curve (AUC) (or its equivalent, the steady-state average concentration) are slightly wider when administered as fixed compared with body weight adjusted dosing. The rationale for a single dose arm:
The rationale for a single dose arm at 120 mg s.c. was to support the dose interval selection in Phase 3. Data from this arm assesses the duration of the response and correlates this with the concentration of drug in the serum at the time when symptoms reappear. This was compared with analogous data from the washout from 240 mg multiple dose arm to ascertain whether longer treatment alters the concentration at the point of symptom reappearance and hence required dosing interval. The totality of the PK, PD and clinical efficacy data, single and multiple dose was analyzed using nonlinear mixed effect PKPD models to generate concentration-response and, from this, dose-response curves from which suitable posologies for registration studies are derived.

Rationale for Duration of Treatment of 20 Weeks

The results of two omalizumab Phase 3 studies (Q4881g and Q4883g) demonstrated that not all responder patients achieve a robust or full response after the first dose. An analysis of the time to a robust clinical response (UAS7≤6) or a full response (UAS7=0) showed that an appreciable fraction of patients who did not respond after the first dose, did progress and achieved a robust or full response after administration of the second and third doses (i.e. after a total treatment of 12 weeks). In addition, examination of the proportion of patients achieving a UAS7≤6 or a symptom free state (UAS=0) across both studies, Q4881g and Q4883g demonstrated a consistent trend for even more patients to achieve an improved health state at Week 24 compared to Week 12. No different safety profile was observed with a longer duration of treatment (Kaplan A, Ledford D, Ashby M, et al (2013) Omalizumab in patients with symptomatic chronic idiopathic/spontaneous urticaria despite standard combination therapy. J Allergy Clin Immunol; 132(1):101-9). In addition, the duration of treatment for the proposed Phase 2b study is 20 weeks to allow sufficient time to evaluate angioedema control using detailed assessments and also explore any differences in clinical response kinetics between subgroups (e.g., CU index+ and CU index −patients). Angioedema was assessed in the omalizumab program primarily as angioedemafree days; newer and more detailed validated assessments (i.e., angioedema activity score [AAS]) are available and these will be applied in this study. As angioedema events are episodic, the longer time to evaluate patients provided a better opportunity to demonstrate control and differentiation from omalizumab and placebo. The CU index is a laboratory test that is commonly used to distinguish patients with an autoimmune component to their CSU and these patients can have specific autoantibodies (e.g., anti-FSRI) that can drive urticaria and may have a different pathophysiology from CU index −patients (Biagtan M J, Viswanathan R K, Evans M D, et al (2011) Clinical utility of the Chronic Urticaria Index. J Allergy Clin Immunol; 127(6):1626-7). CU index+patients compose up to 30% of the CSU population based on the Phase 3 data from omalizumab.

To that end, the selected duration of treatment of 20 weeks in this study was the minimum duration of exposure needed to optimally assess a statistically and clinically meaningful impact on CSU outcomes (Kaplan A, Ledford D, Ashby M, et al (2013) Omalizumab in patients with symptomatic chronic idiopathic/spontaneous urticaria despite standard combination therapy. J Allergy Clin Immunol; 132(1):101-9; Maurer M, Magerl M, Metz M, et al (2013) Revisions to the international guidelines on the diagnosis and therapy of chronic urticaria. J Dtsch Dermatol Ges.). Although this was shorter than the 24 week treatment duration used with omalizumab in Q4881g and Q4883g, it was sufficient for QGE031 based upon the understanding that IgE suppression will occur more rapidly and to a higher level of suppression than observed with omalizumab.

Overall, the study design carefully used available safety and clinical activity data from previous studies with QGE031 and omalizumab, minimized exposure to placebo, and optimized the probability of clinical benefit within the context of a Phase 2 design.

Rationale for Choice of Comparator

All patients, regardless of what treatment arm they were randomized to, received standard of care therapy as background medication. Additionally, patients had access to H1-AH rescue medication.

Placebo was used in this study for the following reasons:
to allow blinding of investigators and patients to their treatment and thereby minimize bias in the evaluation of safety and efficacy assessments,
to allow assessment of the improvement in terms of CSU control for patients with disease not controlled by background medication who are treated with QGE031, in comparison to those continuing solely on background medication, and
to allow the assessment of safety of QGE031 on top of background medication compared to background medication alone
   to maintain the double-blind for lower doses of QGE031 treatment arms as these were administered as 0.2 mL and 0.6 mL respectively. Patients in this treatment arms will receive two injections of matched volume, one active and one placebo.

Omalizumab was selected as an active comparator for the following reasons:
it is the only drug in the same class as QGE031 currently approved as add-on therapy in CSU patients with inadequate response to H1-AH, and
comparison of QGE031 safety and efficacy versus omalizumab assisted in the Sponsor's decision whether there was sufficient potential benefit to patients to warrant further development of QGE031.

TABLE 3
Number of study drug administrations

| | Omalizumab | QGE 031 | Placebo | |
|---|---|---|---|---|
| Treatment Arm | (150 mg/1.2 mL) | (120 mg/1 mL) | (0 mg/1 mL) | Regimen |
| Arm A: | 2 × 1.2 mL | 0 | 0 | q4week |
| Active comparator | | | | |
| (omalizumab 300 mg) | | | | |
| Arm B: | 0 | 2 × 1 mL | 0 | q4week |
| Active drug | | | | |
| (QGE 240 mg) | | | | |
| Arm C: | 0 | 1 × 0.6 mL | 1 × 0.6 mL | q4week |
| Active drug | | | | |
| (QGE 72 mg) | | | | |
| Arm D: | 0 | 1 × 0.2 mL | 1 × 0.2 mL | q4week |
| Active drug | | | | |
| (QGE 24 mg) | | | | |
| Arm E: | 0 | 1 × 1 mL once | 1 × 1 mL once | q4week |
| Active drug | | (Visit 101 only) | (Visit 101) | |
| (QGE 120 mg) | | | 2 × 1 mL four times | |
| | | | (Visits 103, 104, 105, 106) | |
| Arm F: Placebo | 0 | 0 | 2 × 0.6 mL | q4week |
| (0 mg) | | | | |

Concomitant Treatment

This study required concurrent use of H1-AH alone (at approved doses as per local health authority guidance or increased doses up to four-fold) or in combination with H2-AH and/or LTRA (montelukast, zafirlukast, pranlukast) as background medication.

Urticaria Patient Daily Diary (UPDD)

UPDD includes UAS7 (itch and hives) for clinical symptoms, use of rescue medication, sleep and activity interference, angioedema occurrence and its management.

Hives Severity Score (HSS)

The wheals (hives) severity score, defined by number of hives, were recorded by the patient twice daily in their eDiary, on a scale of 0 (none) to 3 (intense/severe) (see Table 4). A weekly score (HSS7) was derived by adding up the average daily scores of the 7 days preceding the visit. The possible range of the weekly score was therefore 0-21.

Complete hives response is defined as HSS7=0.

TABLE 4
Hives Severity Score

| Score | Wheals (Hives) |
|---|---|
| 0 | None |
| 1 | Mild (1-6 hives/12 hours) |
| 2 | Moderate (7-12/12 hours) |
| 3 | Severe (>12 hives/12 hours) |

When either the morning or evening score was missing, the non-missing score for that day (morning or evening) was used as the daily score. When one or more of the daily scores were missing, the following principles were applied to handle the missing data:

If a patient had at least 4 non-missing daily scores within the 7 days prior to the study visit, the weekly score was calculated as the sum of the available eDiary scores in that week, divided by the number of days that have a non-missing diary score, multiplied by 7. If there were less than 4 non-missing daily scores within the prior 7 days, then the weekly score was missing for the week.

Itch Severity Score (ISS)

The severity of the itch was recorded by the patient twice daily in their eDiary, on a scale of 0 (none) to 3 (intense/severe) (see Table 5). A weekly score (ISS7) is derived by adding up the average daily scores of the 7 days preceding the visit. The possible range of the weekly score is therefore 0-21. Partially missing diary entries were handled in the same way as described for the hives severity score.

Complete itch response is defined as ISS7=0.

TABLE 5
Itch Severity Score

| Score | Pruritus (Itch) |
|---|---|
| 0 | None |
| 1 | Mild (minimal awareness, |
| | easily tolerated) |
| 2 | Moderate (definite awareness, |
| | bothersome but tolerable) |
| 3 | Severe (difficult to tolerate) |

The Weekly Urticaria Activity Score (UAS7)

The UAS7 was the sum of the HSS7 score and the ISS7 score. The possible range of the weekly UAS7 score was 0-42.

Complete UAS7 response was defined as UAS7=0.

Sleep Interference Score

Sleep interference was assessed by the patient, once daily in the morning in the eDiary. It was scored on a scale from 0 to 3 (see Table 6).

TABLE 6
Sleep interference score

| Score | Sleep interference |
|---|---|
| 0 | No interference |
| 1 | Mild, little interference with sleep |
| 2 | Moderate, awoke occasionally, |
| | some interference with sleep |
| 3 | Substantial, woke up often, |
| | severe interference with sleep |

Activity Interference Score

Activity interference was assessed by the patients on a scale of 0 to 3 (see Table 7), once daily in the evening in the eDiary. Daily activities could include work, school, sports, hobbies and activities with friends and family.

TABLE 7
Activity interference score

| Score | Activity interference |
|---|---|
| 0 | No interference |
| 1 | Mild, little interference |
| | with daily activities |
| 2 | Moderate, some interference |
| | with daily activities |
| 3 | Substantial, severe interference |
| | with daily activities |

A total of 382 patients were included. The primary objective of the study was achieved, with ligelizumab (QGE031) demonstrating a dose-response relationship with respect to complete hives response rates at Wk12 (p<0.001). HSS7=0 response rates at Wk12 were 30%, 51%, and 42% for ligelizumab 24, 72, and 240 mg, respectively, vs. 26% for omalizumab, and 0% for PBO. These responses were maintained up to Wk20, (26%, 51%, and 45% for ligelizumab 24, 72, and 240 mg, respectively, vs. 34% for omalizumab, and 9% for PBO). High UAS7=0 and DLQI=0-1 response rates were observed as early as Wk4; more patients were symptom-free (UAS7=0) and reported marked improvement of their quality of life (DLQI=0-1) with ligelizumab 72 and 240 mg vs. omalizumab throughout the 20-wk treatment period (Table 8). Ligelizumab was well-tolerated and the safety profile was comparable with that of omalizumab.

TABLE 8
The proportion of complete responders (UAS7 = 0) and those
with DLQI = 0-1 responses at Wk 4, 12 and 20

| | | Ligelizumab | Ligelizumab | Ligelizumab | Omalizumab | |
|---|---|---|---|---|---|---|
| | | 24 mg | 72 mg | 240 mg | 300 mg | Placebo |
| | Week | (n = 43) | (n = 84) | (n = 85) | (n = 85) | (n = 43) |
| Complete | 4 | 23% | 30% | 29% | 20% | 0% |
| UAS7 | 12 | 30% | 44% | 40% | 26% | 0% |
| responders | 20 | 19% | 39% | 40% | 31% | 5% |
| DLQI = 0-1 | 4 | 33% | 41% | 49% | 33% | 10% |
| resonses | 12 | 43% | 61% | 55% | 45% | 33% |
| | 20 | 32% | 67% | 56% | 45% | 23% |

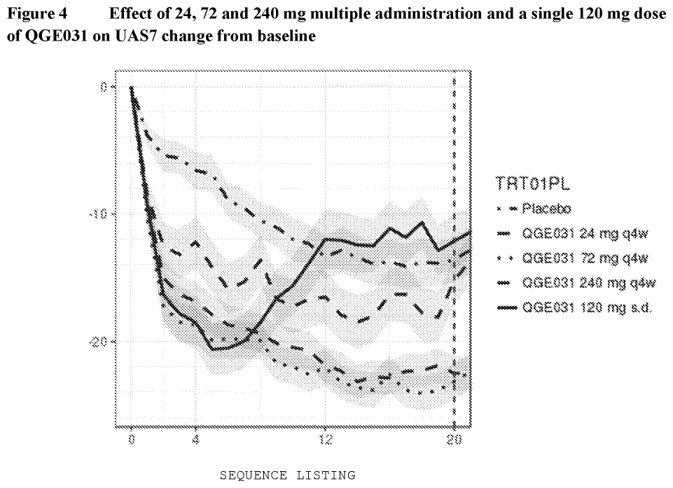

For the first 4-6 weeks of the trial, there was no significant difference in patient response to 72, 120 or 240 mg ligelizumab (FIG. 4). As with the UAS7 complete responders and DLQI=0-1 responses described above, the response to the 24 mg dose was less the three higher doses but still better than placebo. After 6 weeks the effect of the 120 mg single administration started to reduce with symptoms returning towards those of the control group.

Conclusion

In patients with moderate to severe CSU, ligelizumab exhibited a clear dose response across multiple endpoints. Compared with omalizumab 300 mg, ligelizumab 24 mg showed comparable and 72, 120 and 240 mg achieved higher efficacy across said multiple endpoints and showed comparable safety.

### Example 2: Ligelizumab Pediatric Investigation Plan: Exposure-Response Analysis in Adult CSU with Simulation-Based Design of Adolescent Dose-Finding

Objectives: In establishing the pediatric dose, due note was made of a significantly reduced potency, i.e. higher EC₅₀ of omalizumab in the adolescent CSU population [EMA 2014. Application II/0048 Assessment report, Section 2.3.4.2 page 10 and Section 2.3.5 page 11. world wide web.ema.europa.eu/docs/en_GB/document_library/EPAR_-_Assessment_Report_-_Variation/human/000606/WC500164453.pdf accessed 17th January 2018.] compared to adults. It could not therefore be assumed that equivalent concentrations of ligelizumab in adolescent and adults would result in equivalent efficacy in adult and adolescent populations. The objective was to design an adequate adolescent study to determine whether the EC50 for adolescent CSU patients was sufficiently different from adults as to demand a different posology.

Methods: Ligelizumab (QGE031) concentrations and urticaria activity scores (UAS7, 7 day sum of daily itch and hives, each with range 0-3) were collected from adult patients treated with 0, 24, 72 and 240 mg every 4 weeks multiple and 120 mg single dose ligelizumab in the Phase 2 study EudraCT 2014-005559-16. Interim data from 295 patients were analyzed with longitudinal pharmacokinetic-pharmacodynamic models for the continuous UAS7 (range 0-42) using NONMEM with importance sampling. The resultant model was used with stochastic simulation-estimation to design an adolescent (age 11-17 years inclusive) study with the ability to detect shifts in EC50 between adolescents and adults. A combination of R-3.2.3, NONMEM 7.3.0 [Bauer R J NONMEM Users Guide Introduction to NONMEM 7.3.0. 2013. Icon Development Solutions, Hanover Md. 21076, USA] and PDx-Pop-5.2 software was used to create analysis datasets, estimate parameters, control the NONMEM runs and post-process results.

Results: The chosen two-compartment pharmacokinetic model described well the drug concentration data. The key exposure parameter, clearance, was 0.85 L/d (residual standard error, RSE, 9.1%) for 80 kg bodyweight with 49% coefficient of between subject variation (BSV). Bodyweight and chronic urticaria index (anti-IgE or anti-IgE-receptor auto-antibodies) were identified as the main covariates impacting clearance, with estimates of 1.0 (power; 35% RSE) for weight and 0.89 (ratio; 36% RSE) for CU index. The chosen continuous UAS7 model had an EC50 of 1.1 μg/mL (38% RSE) with very large estimated BSV (1405%) and a steep Hill coefficient of 5.72 (0.75% RSE). Visual prediction checks were deemed sufficient to initiate the adolescent study design process over a number of study options. To maintain numerical stability the BSV on EC50 was reduced from that estimated to <=300%, with a sensitivity analysis included to investigate the impact. The design chosen specified three arms: placebo, 24 and 120 mg every 4 weeks with a treatment duration of 16 weeks and follow-up to 40 weeks. The placebo patients should cross to 120 mg after 8 weeks.

Conclusions: Despite highly variable data, the exposure-UAS7 response model was able to detect and describe reasonably well placebo and ligelizumab dose-related changes over time. Stochastic simulation-estimation indicated that 24 mg q4w ligelizumab should be used together with a higher 120 mg q4w dose in the prospective adolescent study. The 24 mg dose was prioritized, as this would generate concentrations in the region of the expected EC50, the optimum point of sensitivity for estimating this parameter. The randomization was therefore uneven, with 20 patients on 24 mg, 10 patients each on 120 mg and placebo. The 120 mg dose, from both the directly treated and crossed-over placebo patients, would enable estimation of the maximum drug effect. Overall, based on 100 simulation-estimations, the procedure indicated that there was approximately 80% chance to detect a 2-fold increase in EC50, the threshold above which a different posology from adults should be considered.

### Example 3: Ligelizumab Retreatment is Highly Effective in Patients with Chronic

Spontaneous Urticaria

Method

In the core Phase 2b trial (NCT02477332), eligible adult patients with moderate to severe CSU (7-day Urticaria Activity Score [UAS7]≥16) were randomised to receive subcutaneous ligelizumab 24, 72 or 240 mg, omalizumab 300 mg, or placebo every 4 weeks (q4w) over 20 weeks. Following the double-blind treatment period, patients entered a 24-week treatment-free period. After washout of the last dose in the core study (Week 32), patients with evidence of disease activity (UAS7≥12) were eligible to enter an open-label, single-arm (ligelizumab 240 mg q4w) extension study. Response after retreatment was assessed with the UAS7; results from the core study are shown for those patients that entered the extension study.

Results

Overall, 70.6% (226/320) of patients entered the extension study. Sustained efficacy (complete responder rates [proportion of patients achieving UAS7=0] and UAS7 change from baseline) was seen following 12 weeks retreatment with ligelizumab 240 mg q4w regardless of dose received during the core study (see Table 9). Similar trends were observed for complete responder rates for HSS7=0 and ISS7=0, as well as mean changes from baseline in HSS7 and ISS7.

TABLE 9
Intra-patients comparison between the core study Week 12 and extension study Week 12^{a}

| | UAS7 = 0 response rate | | UAS7 mean change from | |
|---|---|---|---|---|
| | (95% confidence interval) | | baseline (standard deviation) | |
| | Core study | Extension | Core study | Extension |
| | Week 12 | Week 12 | Week 12 | Week 12 |
| Core study treatment | (N = 51) | (N = 46) | (N = 51) | (N = 46) |
| Ligelizumab 72 mg | 47.1% (32.9, 61.5) | 52.9% (38.5, 67.1) | −21.6 (13.85) | −22.7 (13.74) |
| Ligelizumab 240 mg | 45.7% (30.9, 61.0) | 52.2% (36.9, 67.1) | −22.0 (13.65) | −21.7 (13.36) |

^{a}All patients enrolled in the extension study who received ligelizumab 240 mg q4w are included in this analysis

Conclusions

Ligelizumab retreatment is highly effective in patients with chronic spontaneous urticaria, who have benefited from initial ligelizumab treatment and relapsed following treatment discontinuation.

### Example 4: Ligelizumab Achieves Sustained Control of Chronic Spontaneous Urticaria Symptoms of Hives, Itch and Angioedema: 1-Year Treatment Results

Background

Ligelizumab achieved greater control of symptoms of hives, itch and angioedema versus omalizumab and placebo in patients with chronic spontaneous urticaria (CSU) inadequately controlled with H₁-antihistamines, alone or combined with H2-antihistamines and/or leukotriene receptor antagonists, up to Week 20 (last treatment at Week 16) in the core Phase 2b study (NCT02477332). Here, we report the efficacy and safety of ligelizumab 240 mg up to 1 year in an open-label, single-arm extension study (NCT02649218) in patients who completed the core study and presented with active disease (7-day Urticaria Activity Score [UAS7]≥12).

Methods

After washout of the last dose in the core study and evidence of disease activity, patients entering the extension study received ligelizumab 240 mg every 4 weeks (q4w) for 52 weeks; further monitoring for a 48-week follow-up is ongoing. Disease activity was assessed with the UAS7. Angioedema occurrence was recorded by patients in the Urticaria Patient Daily Diary starting 7 days before the baseline visit (i.e. the visit prior to first administration of ligelizumab in the extension study); at all other visits it was reported for the previous 7 days.

Results

From the core study population, 70.6% (226/320) of patients entered the extension study, with 88.9% (201/226) completing 1 year of open-label treatment. Complete symptom control (UAS=0) was achieved in 35.4% of patients after the first dose of ligelizumab (Week 4). Complete responses were sustained and over 50% of patients achieved UAS7=0 at the end of Week 52. Throughout the one-year treatment period, 75.8% of patients (95% confidence interval [69.9%, 81.3%]) cumulatively experienced complete symptom control at least once by the end of Week 52 based on the Kaplan-Meier method. Angioedema was reported by 33.2% of patients at baseline in the extension phase; this reduced to 10.8% at Week 4. Improvements in the proportion of patients reporting angioedema were sustained up to Week 52, at which time 93.0% of patients were angioedema-free. No new or unexpected safety signals were observed during 1-year of treatment in the extension study.

Conclusion

A high rate of early onset, complete control of hives and itch (UAS7=0), and angioedema was achieved and sustained with ligelizumab 240 mg q4w treatment for 52 weeks in patients with CSU inadequately controlled with standard of care including H₁-antihistamines.

What is claimed is:

1. A method of treating chronic spontaneous urticaria (CSU), comprising subcutaneously (SC) administering to a patient in need thereof a dose of about 24 mg-about 240 mg of an anti-IgE antibody, or an antigen binding fragment thereof, during week 0, and thereafter SC at a dose of about 24 mg-about 240 mg monthly (every 4 weeks), beginning during week 4;
wherein the anti-IgE antibody or antigen binding fragment thereof comprises: a variable light chain region (V_{L}) comprising CDRL1, CDRL2, and CDRL3 and a variable heavy chain region (V_{H}) comprising CDRH1, CDRH2, and CDRH3, wherein CDRL1 consists of SEQ ID NO:3, CDRL2 consists of SEQ ID NO:4, CDRL3 consists of SEQ ID NO:5, CDRH1 consists of SEQ ID NO:6, CDRH2 consists of SEQ ID NO:7, and CDRH3 consists of SEQ ID NO:8.

2. The method according to claim 1, wherein the V_{H} comprises the amino acid sequence set forth as SEQ ID NO:2 and the V_{L} comprises the amino acid sequence set forth as SEQ ID NO:1, wherein the antibody binds specifically to IgE.

3. The method according to claim 1, wherein the anti IgE antibody or antigen binding fragment thereof is a human antibody to human IgE

4. The method according to claim 1, wherein the human antibody to human IgE is ligelizumab.

5. The method according to B, wherein the dose of the ligelizumab antibody or antigen binding fragment is about 24 mg.

6. The method according to 1, wherein the dose of the ligelizumab antibody or antigen binding fragment is about 72 mg.

7. The method according to 1, wherein the dose of the ligelizumab antibody or antigen binding fragment is about 120 mg.

8. The method according to 1, wherein the dose of the ligelizumab antibody or antigen binding fragment is about 240 mg.

9. The method according to claim 1, comprising administering the ligelizumab antibody or antigen binding fragment thereof SC at a dose of about 24 mg during week 0, and thereafter SC at a dose of about 24 mg every four weeks, beginning during week 4.

10. The method according to claim 1, comprising administering the ligelizumab antibody or antigen binding fragment thereof SC at a dose of about 72 mg during week 0, and thereafter SC at a dose of about 72 mg every four weeks, beginning during week 4.

11. The method according to claim 1, comprising administering the ligelizumab antibody or antigen binding fragment thereof SC at a dose of about 120 mg during week 0, and thereafter SC at a dose of about 120 mg every four weeks, beginning during week 4.

12. The method according to claim 1, comprising administering the ligelizumab antibody or antigen binding fragment thereof SC at a dose of about 240 mg during week 0, and thereafter SC at a dose of about 240 mg every four weeks, beginning during week 4.

13. The method according to any of the above claims, wherein said patient achieves a sustained response as measured by complete hives response (Hives Severity Score [HSS7]=0) at week 12 or UAS7=0 and Dermatology Life Quality Index (DLQI)=0-1 at weeks 4, 12 and 20.

14. The method according to any of the above claims, wherein, prior to treatment with the ligelizumab antibody or antigen binding fragment, the patient has been previously treated with a systemic agent for CSU.

15. The method according to claim 14, wherein the systemic agent is selected from the group consisting of H1-antihistamines (H1-AH), H2-AH, and a leukotriene receptor antagonist (LTRA) and combinations thereof.

16. The method according to any of claims 1-13 or 15, wherein, prior to treatment with the IgE antibody or antigen binding fragment, the patient has not been previously treated with a systemic agent for CSU.

17. The method according to any of the above claims, wherein the patient has moderate to severe CSU.

18. The method according to any of the above claims, wherein the patient is an adult.

19. The method according to any of the above claims, wherein the patient is an adolescent.

20. The method according to any of the above claims, wherein the ligelizumab antibody or antigen binding fragment is disposed in a pharmaceutical formulation, wherein said pharmaceutical formulation further comprises a buffer and a stabilizer.

21. The method according to any of the above claims, wherein the pharmaceutical formulation is in liquid form.

22. The method according to any of the above claims, wherein the pharmaceutical formulation is in lyophilized form.

23. The method according to any of the above claims, wherein the pharmaceutical formulation is disposed within pre-filled syringes, vials, injection pens, or autoinjectors.

24. The method according to any of the above claims, wherein the dose of the ligelizumab antibody or antigen binding fragment is about 24 mg, wherein the pharmaceutical formulation is disposed within means for administering selected from the group consisting of a pre-filled syringe, an injection pen, and an autoinjector, wherein said means is disposed within a kit, and wherein said kit further comprises instructions for use.

25. The method according to any of the above claims, wherein the dose of the ligelizumab antibody or antigen binding fragment is about 72 mg, wherein the pharmaceutical formulation is disposed within an autoinjector or a pre-filled syringe, wherein said autoinjector or pre-filled syringe is disposed within a kit, and wherein said kit further comprises instructions for use.

26. The method according to any of the above claims, wherein the dose of the ligelizumab antibody or antigen binding fragment is about 120 mg, wherein the pharmaceutical formulation is disposed within an autoinjector or a pre-filled syringe, wherein said autoinjector or pre-filled syringe is disposed within a kit, and wherein said kit further comprises instructions for use.

27. The method according to any of the above claims, wherein the dose of the ligelizumab antibody or antigen binding fragment is about 240 mg, wherein the pharmaceutical formulation is disposed within autoinjectors or pre-filled syringes, wherein said autoinjectors or pre-filled syringes are disposed within a kit, and wherein said kit further comprises instructions for use.

28. The method according to any of the above claims, wherein the dose is 120 mg, which is administered as a single subcutaneous administration in a total volume of 1 ml from a formulation comprising 120 mg/ml of the IgE antibody or antigen binding fragment.

29. The method according to any of the above claims, wherein the dose is 240 mg, which is administered as a single subcutaneous administration in a total volume of 2 ml from a formulation comprising 120 mg/ml of the IgE antibody or antigen binding fragment.

30. The method according to any of claims 1-3, wherein the anti-IgE antibody or antigen binding fragment has a Tₘₐₓ of about 2-14 days.

31. The method according to any of claims 1-3 and 30, wherein the anti-IgE antibody or antigen binding fragment has an absolute bioavailability of about 47%-about 100%.

32. The method according to any of claims 1-3 and 30-31, wherein the anti-IgE antibody or antigen binding fragment is of the IgG isotype.


SEQUENCE LISTING (8 sequences)

SEQ ID NO: 1 - protein, 107 aa, Artificial Sequence
  Description of Artificial Sequence Synthetic polypeptide
  LIGHT CHAIN VARIABLE REGION OF CLONE CL-2C
EIVMTQSPATLSVSPGERATLSCRASQSIGTNIHWYQQKPGQAPRLLIYYASESISGIPARFSGSGSGTEFTLTISSLQSEDFAVYYCQQSWSWPTTFGGGTKVEIK

SEQ ID NO: 2 - protein, 123 aa, Artificial Sequence
  Description of Artificial Sequence Synthetic polypeptide
  HEAVY CHAIN VARIABLE REGION OF CLONE CL-2C
QVQLVQSGAEVMKPGSSVKVSCKASGYTFSWYWLEWVRQAPGHGLEWMGEIDPGTFTTNYNEKFKARVTFTADTSTSTAYMELSSLRSEDTAVYYCARFSHFSGSNYDYFDYWGQGTLVTVSS

SEQ ID NO: 3 - protein, 11 aa, Artificial Sequence
  Description of Artificial Sequence Synthetic peptide
  TES-C21 CDRL1 SEQUENCE (TABLE 1)
RASQSIGTNIH

SEQ ID NO: 4 - protein, 7 aa, Artificial Sequence
  Description of Artificial Sequence Synthetic peptide
  TES-C21 CDRL2 SEQUENCE (TABLE 1)
YASESIS

SEQ ID NO: 5 - protein, 9 aa, Artificial Sequence
  Description of Artificial Sequence Synthetic peptide
  CDRL3 VARIANT SEQUENCE #2 (TABLE 1)
QQSWSWPTT

SEQ ID NO: 6 - protein, 5 aa, Artificial Sequence
  Description of Artificial Sequence Synthetic peptide
  CDRH1 VARIANT SEQUENCE #1 (TABLE 1)
WYWLE

SEQ ID NO: 7 - protein, 17 aa, Artificial Sequence
  Description of Artificial Sequence Synthetic peptide
  CDRH2 VARIANT SEQUENCE #2 (TABLE 1)
EIDPGTFTTNYNEKFKA

SEQ ID NO: 8 - protein, 14 aa, Artificial Sequence
  Description of Artificial Sequence Synthetic peptide
  TES-C21 CDRH3 (TABLE 1)
FSHFSGSNYDYFDY